(19) United States
(12) Patent Application Publication
(10) Pub. No.: US 2015/0073716 A1
(43) Pub. Date: Mar. 12, 2015

(54) CROP LOSS DETERMINATION

(71) Applicant: Superior Edge, Inc., Mankato, MN (US)

(72) Inventor: Jerome Dale Johnson, Waterville, MN (US)

(73) Assignee: Superior Edge, Inc., Mankato, MN (US)

(21) Appl. No.: 14/021,745

(22) Filed: Sep. 9, 2013

(51) Int. Cl. G01N 33/00

(52) U.S. Cl. CPC G01N 33/0098
USPC 702/19

(57) ABSTRACT

In one example, a method includes receiving, by a crop loss determination generator (CLDG) executing on a computing device, data for a region of interest that includes growing crops. The data for the region of interest includes at least one of field data, crop data, and geographic data. The method further includes determining, by the CLDG and based on the received data for the region of interest, that a crop loss status of the growing crops within the region of interest reflects nonconformance with acceptable crop loss criteria. The crop loss status includes an indication of at least one of an extent and a scope of crop loss of the growing crops within the region of interest. The method further includes outputting, by the CLDG and in response to determining that the crop loss status reflects nonconformance with the acceptable crop loss criteria, at least one alert.

### BACKGROUND

The present invention relates to computing devices, and more particularly to computing devices for use in determining a status of growing crops.

Agricultural crops may be subjected to various types of impactful factors, such as adverse weather conditions (e.g., flooding, hail, and the like), pests, and diseases. Such factors can negatively impact the quality of a harvested crop, and hence, the resulting yield and income for a person managing the crop. Various forms of crop insurance (e.g., hail insurance, flood insurance, or other types of insurance) can be purchased to indemnify the purchaser against a corresponding loss. The extent and scope of the crop loss can be estimated to determine the indemnity payment, such as by manually inspecting portions of the affected crop and extrapolating the findings to the entire field based on the visual inspection. As another example, a perimeter of an impacted crop can be visually inspected (e.g., by driving the perimeter of an impacted field) to create an assumptive assessment of the entire crop.

Such assumptive and estimated loss assessments, however, may not accurately reflect the actual crop loss. As such, resulting indemnity payments may not be proportionate to the actual loss. Inaccurate indemnity payments can negatively impact the agricultural and insurance industries, such as by inaccurately covering a loss of the insured or by increasing the cost of crop insurance.

### SUMMARY

In one example, a method includes receiving, by a crop loss determination generator (CLDG) executing on a computing device, data for a region of interest that includes growing crops. The data for the region of interest includes at least one of field data, crop data, and geographic data. The method further includes determining, by the CLDG and based on the received data for the region of interest, that a crop loss status of the growing crops within the region of interest reflects nonconformance with acceptable crop loss criteria. The crop loss status includes an indication of at least one of an extent and a scope of crop loss of the growing crops within the region of interest. The method further includes outputting, by the CLDG and in response to determining that the crop loss status reflects nonconformance with the acceptable crop loss criteria, at least one alert.

In another example, a system includes a computing device that includes at least one processor and a crop loss determination generator (CLDG) executable by the at least one processor of the computing device. The CLDG is configured to receive data for a region of interest that includes growing crops. The data for the region of interest includes at least one of field data, crop data, and geographic data. The CLDG is further configured to determine, based on the received data for the region of interest, that a crop loss status of the growing crops within the region of interest reflects nonconformance with acceptable crop loss criteria. The crop loss status includes an indication of at least one of an extent and a scope of crop loss of the growing crops within the region of interest. The CLDG is further configured to output, in response to determining that the crop loss status reflects nonconformance with the acceptable crop loss criteria, at least one alert.

In a further example, a computer-readable storage medium is encoded with instructions that, when executed, cause at least one processor of a computing device to receive data for a region of interest that includes growing crops. The data for the region of interest includes at least one of field data, crop data, and geographic data. The computer-readable storage medium is further encoded with instructions that, when executed, cause the at least one processor of the computing device to determine, based on the received data for the region of interest, that an agronomic status of the growing crops within the region of interest reflects nonconformance with acceptable criteria. The agronomic status includes an indication of at least one of an extent and a scope of crop loss of the growing crops within the region of interest. The computer-readable storage medium is further encoded with instructions that, when executed, cause the at least one processor of the computing device to output, in response to determining that the agronomic status reflects nonconformance with the acceptable criteria, at least one alert.

In some examples, a method includes receiving, by a field growth determination generator (FGDG) executing on a computing device, data for a region of interest that includes growing biological matter. The data for the region of interest includes at least one of field data, agronomic data, and geographic data. The method further includes determining, by the FGDG and based on the received data for the region of interest, that an agronomic status of the biological matter within the region of interest reflects nonconformance with acceptable criteria. The method further includes outputting, by the FDDG and in response to determining that the agronomic status reflects nonconformance with the acceptable criteria, at least one alert.

### BRIEF DESCRIPTION OF THE DRAWINGS

FIG. 1 is a block diagram illustrating an example crop loss determination system, in accordance with one or more aspects of this disclosure.

FIG. 2 is a block diagram illustrating further details of one example of a server device shown in FIG. 1.

FIG. 3 is a block diagram illustrating further examples of a database illustrated in FIG. 1.

FIG. 4 illustrates an example geographic information system (GIS) that can be used to determine a crop loss status.

FIG. 5 is a flow diagram illustrating example operations to determine a crop loss status and automatically output at least one alert.

FIG. 6 is a flow diagram illustrating further details of the operations of FIG. 5.

FIG. 7 is a flow diagram illustrating further details of the operations of FIG. 5.

FIG. 8 is a flow diagram illustrating further details of the operations of FIG. 5.

FIG. 9 is a flow diagram illustrating further details of the operations of FIG. 5.

FIG. 10 illustrates a table that represents an example scoring matrix for use in a method of determining a crop loss status of growing crops within a region of interest.

FIG. 11 illustrates a table that represents another embodiment of an example scoring matrix for use in a method of determining a crop loss status of growing crops within a region of interest.

FIG. 12 illustrates a table that represents example calculations that can be used to determine a crop loss status of growing crops within a region of interest.

FIG. 13 illustrates example images that can be used to determine a crop loss status for a region of interest.

FIG. 14 illustrates and example user interface including an alert.

FIG. 15 illustrates an example user interface that can be used to review information related to crop loss.

### DETAILED DESCRIPTION

According to techniques described herein, a computing device can dynamically analyze various forms of data associated with agricultural crops to determine the scope and extent of damage to the crop. For instance, a computing device implementing techniques of this disclosure can receive data of various types from multiple sources, such as image data from a camera or other image sensor, weather data from one or more data feeds (e.g., public and/or private data sources), data entered via a user interface communicatively coupled to the computing device, or other types of data. The computing device can analyze the received data to determine a crop loss status of growing crops within a region of interest (e.g., a field of crops) and can automatically provide one or more alerts (e.g., email, SMS message, voice message, alerts provided via a graphical user interface, or other types of alerts) in response to determining that the crop loss status reflects nonconformance with acceptable crop loss criteria. In this way, techniques described herein can improve the accuracy and efficiency of crop loss determinations. Moreover, a computing device implementing techniques of this disclosure can provide timely alerts of crop loss to interested parties, such as producers (e.g., farmers), insurance carriers, buyers of agricultural products, agricultural landlords and bankers, and the like. Accordingly, the computing device can possibly enable such parties to take corrective action to minimize further loss.

While described herein with respect to determining crop loss status of growing crops within a region of interest, techniques of this disclosure are not so limited. For instance, in certain examples, rather than determine a crop loss status, a computing device implementing techniques of this disclosure can determine a growth status. Such growth status and/or loss status can be referred to as an agronomic status. Similarly, while the techniques are described herein with respect to a status of growing crops, in certain examples, a computing device as described in this disclosure can determine an agronomic status for any growing biological matter. Accordingly, while described with respect to crop loss analysis, the techniques described herein can, in certain examples, be applied to determine one or more of a loss and growth status of biological matter (e.g., including growing crops, such as agricultural crops) within a region of interest.

An agricultural crop can be subjected to multiple types of impactful factors, such as pests, diseases, adverse weather conditions including wind, flooding, hail, killer frost, drought, or other types of factors that can impact the quality of a harvested crop. Such factors can decrease the quality and/or quantity of the harvested crop and, therefore, the resulting yield and income received by the person managing the crop. These factors that negatively impact the quality of the crop can be safeguarded against through the use of various forms of crop insurance, including, for example, hail insurance. Other types of crop insurance can include revenue-based insurance, yield-based insurance, or combinations of the two. Crop insurance is generally purchased or committed to prior to planting a crop, and the level of coverage is determined based upon the type of crop planted, its intended post-harvest use, historical production history of the field, and the level of coverage desired. Often, the extent and the scope of the crop loss is measured against production history of previous years on that same field (typically referred to as the Actual Production History, or “APH”). The “extent” of crop loss can refer to the severity of the damage to the crop, such as the percentage of an amount of crop that is lost within a region of interest (e.g., a percentage of crop loss within a particular number of acres). The “scope” of crop loss can refer to the area of crop affected, such as a quantity of acres affected.

Various conventional methods may be used to determine the scope and extent of crop loss. For instance, following the occurrence of a negatively impactful factor upon a crop, an adjuster may typically assess the damage. As one example, an adjuster may travel (e.g., walk, drive, etc.) through the field to inspect the crop at various points and extrapolate findings to an entire field based on a visual inspection of random points within that field. Another, and possibly less-thorough example, includes driving along the perimeter of an impacted crop and creating an assumptive assessment of the entire crop based on that visual inspection. As yet another example, “weigh tickets” may be used to determine crop loss. Weigh tickets may be produced when the farmer harvests the crop and then weighs it. This technique can be fairly successful in determining accurate crop loss, but is dependent on the farmer actually planting and also harvesting the crop, which may not be practical or desirable on certain occasions. For instance, a farmer can apply for “prevented planting” insurance, if, for example, field conditions do not allow for normal and/or typical plant growth. As one example, a field may be too flooded to plant. As another example, a remaining growing season may be too short to plant a particular crop due to, for example, a late spring (e.g., frost leaving the ground later than normal). In such examples, it may not be practical and/or cost effective for a farmer to plant a crop, thereby prompting a claim against a prevented planting insurance policy.

Similarly, in examples when a crop is planted but damaged due to, for example, adverse weather conditions (e.g., hail, flooding, frost, and the like), a farmer may not want to expend the resources to harvest a crop if the damage is too great. In these scenarios, it may not be practical to use weigh tickets to determine the scope and extent of crop loss. Moreover, the accuracy of using weigh tickets is also dependent on the harvested crop being assigned to the right field, and both the yield monitors that are internal to combines and the weigh ticket scales themselves being calibrated. In short, weigh tickets can present logistical impediments that can prevent them from being useful and reliable in certain cases. In such examples, a system implementing techniques of this disclosure can determine a crop loss status or, conversely, a field growth status of growing crops within a region of interest.

A person managing the crop (e.g., a farmer), also oftentimes negotiates with an adjuster to reach an agreement regarding the extent and scope of the damage upon the crop. The insurance assessment typically includes an identification of the size of the area impacted (e.g., the scope of crop loss) and the percentage of loss upon that area (e.g., the extent of crop loss). Conventional techniques that determine crop loss based on random sample inspections extrapolated into assumptive estimates of the entire crop may be untimely, inaccurate, and may be prone to human error and fraud. Similarly, such conventional techniques may not provide sufficient evidence that can be used when audits (e.g., government audits, insurance audits, and the like) are performed. Accordingly, such conventional assumptive and estimated loss assessments may not accurately reflect the loss, and may result in indemnity payments that are not proportionate to the loss.

Inaccurate estimates of crop insurance indemnity payments can ultimately result in negative consequences to both the agricultural industry and the insurance industry, by either not properly covering a loss of the insured or by creating an insurance market that costs more for farmers and the United States government through its crop insurance subsidy program. Additionally, the government may require that large loss claims (e.g., loss claims corresponding to a monetary value greater than a threshold value, such as $100,000, $200,000, or other threshold values) are audited to ensure the accuracy of the claim. These audits are often time-consuming and can often be delayed due to the overabundance of these claims during the harvesting season. The resulting backlog can result in a time delay between when a farmer applies for an indemnity payment and a time when the audit is performed. During such time, the status of the crop loss can change, thereby resulting in an inaccurate loss assessment and corresponding indemnity payment.

There are a large number of conditions and events that can result in a loss of crop yield, quality, and revenue. Moreover, factors can vary considerably from region to region, field to field, and crop to crop. There are possibly millions of insured fields, with hundreds of crops, all with various value and subject to a wide variety of conditions. A computing device implementing techniques described herein can help to improve the efficiency and precision by which crop loss is determined. For example, according to techniques of this disclosure, a computing device can receive data (e.g., image data) regarding a region of interest (e.g., a field of crops) from a remotely piloted vehicle (RPV) equipped with an image sensor (e.g., a camera). Such RPVs can be flown frequently, at will, and at a low cost. In addition to using RPVs to capture visual data, sometimes multiple times over the crop cycle, the computing device may receive and analyze data from multiple other sources, alone or in combination, to create a more meaningful, precise, and accurate crop loss assessment. Examples of such data can include, but are not limited to, meteorological data, weather data, geographic information systems (GIS) data, planting equipment data, harvesting equipment data, and manually ascertained data. By analyzing multiple types of data received from multiple sources, a computing device implementing techniques described herein can increase the accuracy of crop loss assessments and the efficiency by which such assessments are determined.

For example, a crop loss determination system implementing techniques of this disclosure can receive multiple types of data from multiple different sources, including in-season data related to agricultural crops within a region of interest (e.g., a field of crops). The crop loss determination system can dynamically analyze the data to determine the scope and extent of damage to a crop, document (e.g., store) the crop loss status, and automatically output alerts and other relevant information concerning that crop loss. In some examples, the crop loss determination system can include a user interface, data feeds, data sources, a communication network, a crop loss determination generator, a database, or one or more other components.

The crop loss determination generator can receive data for the region of interest from a variety of sources, such as from one or more of a user interface, a database, a data feed, an Internet-based data source, a remote sensor (e.g., an RPV), a social network, and equipment used by farmers. In some examples, the crop loss determination generator can receive such data via a communication network, such as the Internet, a cloud computing network, a cellular network, a local area network (LAN), a wide area network (WAN), a wireless LAN (WLAN), or other types of networks.

The user interface, executable by a computing device, can be configured to receive alerts, analyses, and statuses from the crop loss determination generator via the communication network. In addition, the user interface can enable a user to interact with the crop loss determination system. For instance, the user interface can be configured to receive information regarding manually ascertained data, such as data manually ascertained by a user and manually input to the user interface, and to provide such data to the crop loss determination generator. Similarly, the user interface can be configured to receive an indication of the crop loss status from the crop loss determination generator and output such status, such as to a user, to one or more computing devices, etc.

In some examples, the crop loss determination system can include a database that is configured to store crop loss information. The database can be communicatively coupled to the crop loss determination generator. The crop loss determination generator can receive data from the database, analyze the received data, and determine a crop loss status for a region of interest (e.g., a field, a portion of a field, and the like). The crop loss determination generator can determine the scope and extent of crop loss for the region of interest based at least in part on the received data. For example, the crop loss determination generator can determine the scope and extent of crop loss within the region of interest based at least in part on image data for the region of interest, such as image data including, for example, reflection data, pattern data, color data, texture data, shape data, shadow data, visible and/or non-visible light spectrum data, chemical image data, hyperspectral image data, and/or electronically modified (e.g., enhanced) image data for the region of interest.

In some examples, the crop loss determination generator can receive one or more other types of data, such as one or more of field data (e.g., soil types and textures), topography data, weather data, harvest equipment data, seed performance data, past crop insurance data, and data from other farmers through what may be described as a social network. The crop loss determination generator can use one or more of the received data to determine the scope and extent of the crop loss. In this way, the crop loss determination generator can determine potential crop loss to enable a user (e.g., a farmer, an insurance adjuster, an auditor, and the like) to properly assess the extent and scope of the loss and establish the appropriate indemnity. The crop loss determination generator can determine a crop loss status with respect to an entire agricultural field or a portion of the field. In some examples, the crop loss generator can determine whether the crop loss status reflects conformance with acceptable crop loss criteria. In certain examples, in response to determining that the crop loss status reflects nonconformance with acceptable crop loss criteria, the crop loss determination generator can output at least one alert. In some examples, in response to determining that the crop loss status does not reflect nonconformance with acceptable crop loss criteria (i.e., reflects conformance with the acceptable crop loss criteria), the crop loss determination generation can refrain from outputting an alert.

Examples of users of the crop loss determination system can include, but are not limited to, farmers, crop insurance agents, adjusters and/or other representatives from a crop insurance carrier, the Risk Management Agency (RMA), governmental agencies with oversight of crop loss, buyers of agricultural products, agricultural landlords and/or bankers, or other persons who have a vested interest and/or responsibility in the growth and outcomes of an agricultural crop. Data incorporated into the crop loss determination system can be received and/or derived from various sources, such as, but not limited to, a user via a user interface, user equipment, remote sensors (e.g., an RPV), Internet-based data sources, other farmers, and/or commercial, governmental, and/or public data sources. Similarly, data incorporated into the crop loss determination system can include various types of data, such as field data (e.g., soil types), weather data, climate data, terrain data (e.g., elevation and/or slope data), agronomic data (e.g., seed genetic data, seed performance characteristics data, plant research data, plant performance data, and the like). As another example, data incorporated into the crop loss determination system can include image data and/or image research data based on, for example, hyperspectral plant imprints and/or optical signatures. For instance, the crop loss determination generator can receive hyperspectral wavelength data for growing crops within the region of interest and can compare the received hyperspectral wavelength data to one or more optical signatures that indicate various stages of plant health (e.g., nitrogen deficiency) and/or characteristic hyperspectral wavelengths associated with various plant conditions responsive to stressors, such as diseases, infestations, and the like.

In some examples, the crop loss determination generator can determine an attribute of received data and can include the received data into a corresponding attribute of the database. For instance, in examples where an attribute of the received data relates to the condition of the condition, the crop loss determination generator can incorporate the received data having the attribute that relates to the condition of the field into a corresponding field condition attribute of the database.

In certain examples, the user interface can receive configuration data (e.g., from a user) that configures (e.g., according to user preferences) how the crop loss determination generator receives and analyzes data, the parameters around how and when the system notifies the user or other designated parties of crop loss, any exclusions that the user desires to be exempt from the analyzed data, and the manner and method by which the user, and/or other designated parties, are to be alerted. The crop loss determination generator can output alerts, which can be received by a user and/or other designated parties via the communication network and the user interface. Examples of such alerts can include text messages, phone messages, voicemail messages, emails, or other types of alerts. In certain examples, an alert can include information such as maps to specify the location, size, and shape of the area where the crop loss has been determined, and/or an indication that the crop loss status does not satisfy (e.g., falls outside) the acceptable crop loss criteria. In some examples, the alert can include a visual analysis in the form of a chart or graph displaying determinations, locations, and comparative or benchmark data.

The crop loss determination generator can receive configuration data (e.g., via the user interface, a file upload, and the like) that specifies data display preferences that can enable a more nuanced view of the crop loss determination data. For instance, a data display configuration parameter can exclude geographic areas within a region of interest that are not included within the crop loss determination area. Such exclusionary configuration parameters can enable a user to remove from consideration data and/or areas of a field that are physically incongruent with the rest of the field (e.g., ditches, rock piles, former building sites, etc.) and that would therefore skew or distort the overall dataset and the resulting determinations. If, in this example, the crop loss determination generator receives configuration data (e.g., from a user via a user interface) that specifies an exclusionary zone within the region of interest due to, for example, information known by the user at the local level, such as the presence of a former building site, a prior manure or fertilizer spill, and the like, the crop loss determination generator can exclude the region defined by the exclusionary zone from the region of interest and hence from the crop loss determination analysis.

The crop loss determination system can receive data over a time period (e.g., a growing season, multiple years, or other time periods) and output a comparison of received data of the same crop in the same field over the time period. Likewise, through the use of social networks, peer users may compare their crop loss with others, including those other users who have crops in relative proximity and therefore are subject to similar environmental conditions (soil types, climate, weather, seed varieties, pests, etc.). In some examples, the user interface can be configured to output underlying data for display, such that a user may be able to personally view the underlying data. The crop loss determination generator can output alerts other interested parties, as designated by configuration parameters defined by, for example, a user via the user interface. Such alerts can help to keep suppliers, buyers, landlords, and others abreast of the in-season crop growth and crop loss.

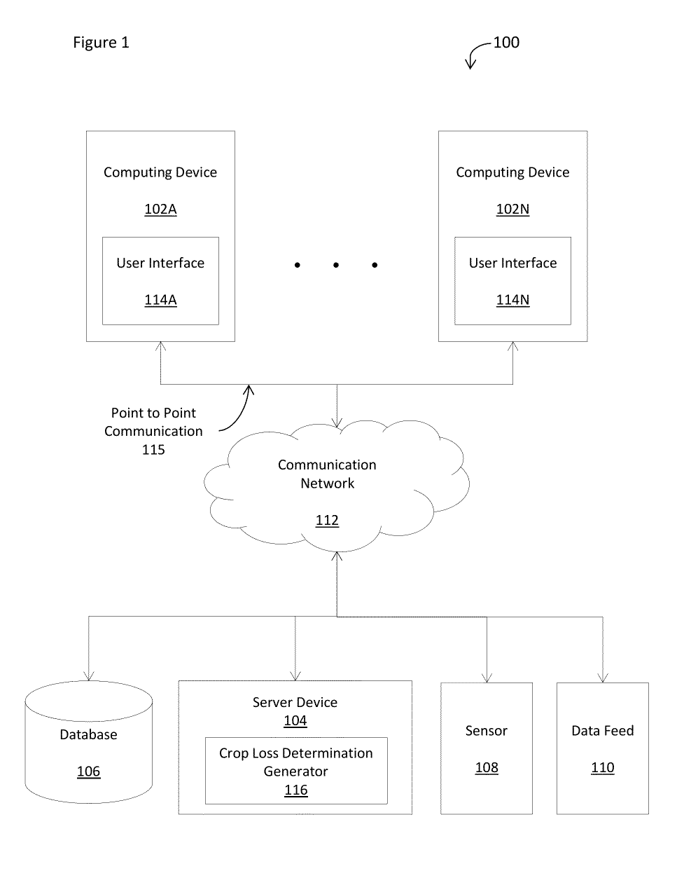

FIG. 1 is a block diagram illustrating an example crop loss determination system 100, in accordance with one or more aspects of this disclosure. As illustrated in FIG. 1, crop loss determination system 100 can include computing devices 102A-102N (collectively referred to herein as “computing devices 102”), server device 104, database 106, sensor 108, data feed 110, and communication network 112. Each of computing devices 102 can include a user interface, illustrated in FIG. 1 as user interfaces 114A-114N, and collectively referred to herein as “user interfaces 114.” Server device 104 can include crop loss determination generator 116.

While illustrated with respect to computing devices 102A-102N, computing devices 102 can include any number of computing devices, such as one computing device 102, two computing devices 102, five computing devices 102, fifty computing devices 102, or other numbers of computing devices 102. Examples of computing devices 102 can include, but are not limited to, portable or mobile devices such as mobile phones (including smartphones), laptop computers, tablet computers, desktop computers, personal digital assistants (PDAs), servers, mainframes, or other computing devices.

Computing devices 102, in certain examples, can include user interfaces 114. For example, computing device 102A can include user interface 114A, executable by one or more processors of computing device 102A, that can enable a user to interact with computing device 102A and crop loss determination system 100 via one or more input devices of computing device 102A (e.g., a keyboard, a mouse, a microphone, a camera device, a presence-sensitive and/or touch-sensitive display, or one or more other input devices). User interfaces 114 can be configured to receive input (e.g., in the form of user input, a document or file, or other types of input) and provide an indication of the received input to one or more components of crop loss determination system 100 via communication network 112.

As illustrated in the example of FIG. 1, communication network 112 communicatively couples components of crop loss determination system 100. Examples of communication network 112 can include wired or wireless networks or both, such as local area networks (LANs), wireless local area networks (WLANs), cellular networks, wide area networks (WANs) such as the Internet, or other types of networks. Although the example of FIG. 1 is illustrated as including one communication network 112, in certain examples, communication network 112 may include multiple communication networks. In addition, as illustrated in FIG. 1, one or more of computing devices 102 can communicate with one another via point-to-point communications 115.

Database 106 can include one or more databases configured to store data related to crop loss determination. For instance, database 106 can include one or more relational databases, hierarchical databases, object-oriented databases, multi-dimensional databases, or other types of databases configured to store data usable by crop loss determination system 100 to determine a crop loss status of growing crops within a region of interest. As an example, and as further described herein, database 106 can include one or more databases configured to store field data, production data, meteorological data, weather data, manually ascertained data, agronomic data, geographic data, crop data, equipment data, configuration data, optical signature data, or other types of data that are retrievable by crop loss determination generator 116 to determine a current crop loss status.

Sensor 108 can include one or more sensors capable of gathering data usable by crop loss determination system 100. For instance, sensor 108 can include one or more of a remote sensor (e.g., a sensor that is physically remote from the region of interest) and an in-field sensor (e.g., a sensor that is physically proximate and/or within the region of interest). As one example, sensor 108 can include an image sensor, such as an image sensor included within a camera device (e.g., a visible-spectrum image sensor, an ultra-violet (UV) image sensor, an infra-red image sensor such as included in a thermal imaging camera, a hyperspectral image sensor, or other types of image sensors) and configured to gather image data for a region of interest, such as a field of growing crops. Such image data can include, but is not limited to, optical signature image data, crop color data (e.g., traditional, red, infrared, green, blue), pattern data, tone data, texture data, shape data, and shadow data.

In certain examples, sensor 108 can include one or more other sensors, such as precipitation sensors (e.g., a rain gauge), light sensors, wind sensors, or other types of sensors. In some examples, sensor 108 can include one or more remote sensors carried by, for example, a remotely piloted vehicle (RPV), an unmanned aerial vehicle (UAV), an aircraft, a satellite, and the like. For instance, sensor 108 may include one or more image sensors included within a camera device carried by an RPV and configured to capture image data for a region of interest (e.g., a field, a portion of a field, a region including a field and its surrounding area, and the like). Such RPVs can be convenient vehicles for obtaining in-season data related to crop condition due in part to their ability to gather data in a timely, quick, scalable, and economical manner.

As illustrated in FIG. 1, one or more components of crop loss determination system 100 can be configured to receive data from data feed 110 (e.g., via communication network 112, point-to-point communications 115, peer-to-peer communication, etc.). Examples of data received by components of crop loss determination system 100 from data feed 110 can include vegetation data, weather data (e.g., temperature data, average temperature data, data indicating events such as thunderstorms, floods, hail, wind storms, etc.), climate data, or other types of data. Data feed 115 may provide data to components of crop loss determination system 100 via various sources, such as commercial, governmental, public and/or fee-based data sources. For instance, such sources can include Internet-based sources, such as the United States Department of Agriculture, the National Oceanic and Atmospheric Administration, the RMA, or other public and/or private data sources. As another example, data feed 110 can provide data to components of crop loss determination system 100 from sources such as combines, planters, sprayers, cultivators, and other equipment used to execute various agricultural practices, as well as academic and/or research organizations, suppliers of crop inputs, buyers of crops, and peer farmers. In some examples, data feed 110 can provide information obtained from a social networking service, such that data feed 110 can provide components of crop loss determination system 100 with information obtained from peer farmers and/or other computing systems.

As illustrated in the example of FIG. 1, crop loss determination system 100 can include server device 104. In certain examples, server device 104 can be substantially similar to computing devices 102, in that server device 104 can be a computing device including one or more processors capable of executing computer-readable instructions stored within memory of server device 104 that, when executed, cause server device 104 to implement functionality according to techniques described herein. For instance, server device 104 can be a portable or non-portable computing device, such as a server computer, a mainframe computer, a desktop computer, a laptop computer, a tablet computer, a smartphone, or other type of computing device. In some examples, although illustrated in FIG. 1 as including one server device 104, crop loss determination system 100 can include multiple server devices 104. For instance, in certain examples, crop loss determination system 100 can include multiple server devices 104 that distribute functionality attributed to server device 104 among the multiple server devices.

As illustrated, server device 104 can include crop loss determination generator (CLDG) 116. CLDG 116 can include any combination of software and/or hardware executable by one or more server devices 104 to determine a growth status and/or a crop loss status according to techniques described herein. As an example, CLDG 116 can receive data for a region of interest that includes growing crops. For instance, CLDG 116 can receive data from one or more of computing devices 102 (e.g., via user interfaces 114), database 106, sensor 108, and data feed 110 via communication network 112, point-to-point communications 115, and the like. The received data can include data usable by CLDG 116 to determine a crop loss status of growing crops within the region of interest. For example, CLDG 116 can receive one or more of field data, production data, weather data, manually ascertained data, geographic data, meteorological data, crop data, equipment data, configuration data, optical signature data, or other types of data.

CLDG 116 can determine, based at least in part on the received data for the region of interest, that a crop loss status of the growing crops within the region of interest reflects nonconformance with acceptable crop loss criteria. As an example, CLDG 116 can determine, based on one or more of the received data, that a crop loss status falls outside a range of acceptable crop loss criteria, such as a range of percentages of crop loss, a range of areas of the region of interest in which crop loss is determined, and the like. In certain examples, CLDG 116 can determine that the crop loss status of the growing crops within the region of interest reflects nonconformance with acceptable crop loss criteria based on a determination, by CLDG 116, that the received data does not satisfy one or more parameters (e.g., is greater than the one or more parameters, greater than or equal to the one or more parameters, falls outside a range of one or more parameters, and the like).

In some examples, rather than determine a crop loss status, CLDG 116 can determine a growth status of biological matter (e.g., including growing crops) within a region of interest. In such examples, CLDG 116 can be referred to as a field growth determination generator. Such a field growth determination generator can determine an agronomic status (e.g., a loss and/or growth status) of biological matter within a region of interest.

A crop loss status can include an indication of at least one of an extent of crop loss (e.g., an indication of a severity of crop loss, such as a percentage of crop loss) and a scope of crop loss (e.g., an indication of an area of the region of interest in which crop loss is determined, such as a number of acres) of the growing crops within the region of interest. In response to determining that the crop loss status reflects nonconformance with the acceptable crop loss criteria, CLDG 116 can output at least one alert. For instance, CLDG 116 can output the at least one alert including one or more email messages, short messaging service (SMS) messages, voice messages, voicemail messages, audible messages, or other types of messages that include an indication of the at least one alert. In certain examples, CLDG 116 can output an alert to user interfaces 114 (e.g., via communication network 112). In some examples, CLDG 116 can determine a distribution list, such as a list of accounts associated with crop loss determination system 100 (e.g., user accounts, accounts associated with one or more other computing systems, etc.), and can output the at least one alert to the list of accounts.

In certain examples, crop loss determination system 100 can include one or more components not illustrated in FIG. 1. For instance, as discussed above, crop loss determination system 100 can include, in some examples, multiple server devices 104 that distribute functionality of server device 104 among the multiple server devices 104. Similarly, one or more illustrated components of crop loss determination system 100 may not be present in each embodiment of crop loss determination system 100. For instance, in certain examples, at least one computing devices 102 and server device 104 may comprise a common device. For example, server device 104 and computing device 102A can, in some examples, be one device that executes both CLDG 116 and user interface 114A.

As one example operation of crop loss determination system 100 of FIG. 1, CLDG 116, executing on one or more processors of server device 104, can receive data for a region of interest, such as a field of growing crops. For instance, CLDG 116 can receive, via communication network 112, the data for the region of interest from one or more of database 106, sensor 108, data feed 110, and computing devices 102 (e.g., via one or more of user interfaces 114). CLDG 116 can determine, based on the received data for the region of interest, that a crop loss status of the growing crops within the region of interest reflects nonconformance with acceptable crop loss criteria, such as criteria that define an acceptable severity and/or scope of crop loss. CLDG 116 can output, in response to determining that the crop loss status reflects nonconformance with the acceptable crop loss criteria, at least one alert. For instance, CLDG 116 can output one or more alerts to one or more of computing devices 102, such one or more alerts that are output to one or more of user interfaces 114, one or more email messages, voice messages, voicemail messages, text messages, SMS messages, or other types of alerts. In certain examples, the one or more alerts can include an indication of a degree by which the crop loss status of the growing crops within the region of interest deviates from the acceptable crop loss criteria. In some examples, the one or more alerts can include an indication of the region of interest and/or a portion of the region of interest (e.g., a portion of the field) that reflects nonconformance with the acceptable crop loss criteria.

In this way, CLDG 116 can dynamically analyze multiple forms of data received from multiple input sources to determine a crop loss status of growing crops within a region of interest. CLDG 116 can automatically output at least one alert in response to determining that the crop loss status reflects nonconformance with acceptable crop loss criteria. Accordingly, CLDG 116 can output timely alerts regarding crop loss that may enable a user, such as a farmer, to take corrective action, such as by replanting one or more portions of a field, to help minimize the scope and extent of the loss. Moreover, by analyzing multiple forms of data, CLDG 116 can increase the accuracy of the determination of the crop loss status, thereby possibly enabling a more accurate indemnity payment corresponding to the loss.

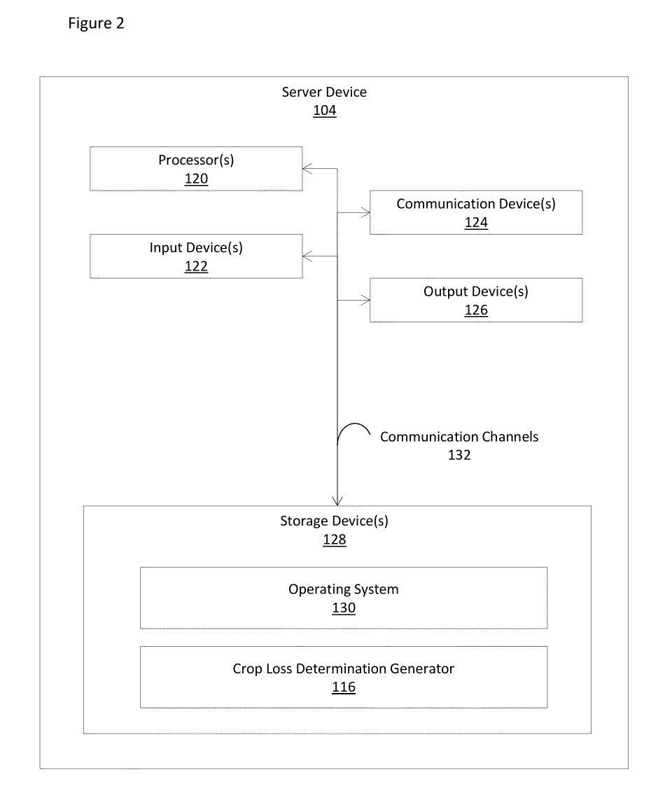

FIG. 2 is a block diagram illustrating further details of one example of server device 104 shown in FIG. 1, in accordance with one or more aspects of this disclosure. FIG. 2 illustrates only one example of server device 104, and many other examples of server device 104 can be used in other examples.

As shown in the example of FIG. 2, server device 104 can include one or more processors 120, one or more input devices 122, one or more communication devices 124, one or more output devices 126, and one or more storage devices 128. As illustrated, server device 104 can include operating system 130 and CLDG 116 that are executable by server device 104 (e.g., by one or more processors 120).

Each of components 120, 122, 124, 126, and 128 can be interconnected (physically, communicatively, and/or operatively) for inter-component communications. In some examples, communication channels 132 can include a system bus, a network connection, an inter-process communication data structure, or any other method for communicating data. As illustrated, components 120, 122, 124, 126, and 128 can be coupled by one or more communication channels 132. Operating system 130 and CLDG 116 can also communicate information with one another as well as with other components of server device 104, such as output devices 126.

Processors 120, in one example, are configured to implement functionality and/or process instructions for execution within server device 104. For instance, processors 120 can be capable of processing instructions stored in storage device 128. Examples of processors 120 can include any one or more of a microprocessor, a controller, a digital signal processor (DSP), an application specific integrated circuit (ASIC), a field-programmable gate array (FPGA), or other equivalent discrete or integrated logic circuitry.

One or more storage devices 128 can be configured to store information within server device 104 during operation. Storage device 128, in some examples, is described as a computer-readable storage medium. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, storage device 128 is a temporary memory, meaning that a primary purpose of storage device 128 is not long-term storage. Storage device 128, in some examples, is described as a volatile memory, meaning that storage device 128 does not maintain stored contents when power to server device 104 is turned off. Examples of volatile memories can include random access memories (RAM), dynamic random access memories (DRAM), static random access memories (SRAM), and other forms of volatile memories. In some examples, storage device 128 is used to store program instructions for execution by processors 120. Storage device 128, in one example, is used by software or applications running on server device 104 (e.g., CLDG 116) to temporarily store information during program execution.

Storage devices 128, in some examples, also include one or more computer-readable storage media. Storage devices 128 can be configured to store larger amounts of information than volatile memory. Storage devices 128 can further be configured for long-term storage of information. In some examples, storage devices 128 include non-volatile storage elements. Examples of such non-volatile storage elements can include magnetic hard discs, optical discs, floppy discs, flash memories, or forms of electrically programmable memories (EPROM) or electrically erasable and programmable (EEPROM) memories.

Server device 104, in some examples, also includes one or more communication devices 124. Server device 104, in one example, utilizes communication device 124 to communicate with external devices via one or more networks, such as one or more wireless networks. Communication device 124 can be a network interface card, such as an Ethernet card, an optical transceiver, a radio frequency transceiver, or any other type of device that can send and receive information. Other examples of such network interfaces can include Bluetooth, 3G, 4G, and WiFi radio computing devices as well as Universal Serial Bus (USB). In some examples, server device 104 can utilize communication device 124 to wirelessly communicate with an external device, such as one or more sensors 108 (illustrated in FIG. 1).

Server device 104, in one example, also includes one or more input devices 122. Input device 122, in some examples, is configured to receive input from a user. Examples of input device 122 can include a mouse, a keyboard, a microphone, a camera device, a presence-sensitive and/or touch-sensitive display, or other type of device configured to receive input from a user.

One or more output devices 126 can be configured to provide output to a user. Examples of output device 126 can include, a display device, a sound card, a video graphics card, a speaker, a cathode ray tube (CRT) monitor, a liquid crystal display (LCD), or other type of device for outputting information in a form understandable to users or machines.

Server device 104 can include operating system 130. Operating system 130 can, in some examples, control the operation of components of server device 104. For examples, operating system 130, in one example, facilitates the communication of CLDG 116 with processors 120, input devices 122, communication devices 124, and/or output devices 126.

CLDG 116 can include program instructions and/or data that are executable by server device 104 to perform one or more of the operations and actions described in the present disclosure. For instance, CLDG 116 can receive data for a region of interest from one or more of communication devices 124 (e.g., from a remote device, such as from one or more of computing devices 102, sensor 108, data feed 110, and/or database 106) and input devices 122 (e.g., a mouse, keyboard, or other input devices). CLDG 116, executing on one or more processors 120, can determine, based on the received data for the region of interest, that a crop loss status of growing crops within the region of interest reflects nonconformance with acceptable crop loss criteria. For instance, CLDG 116 can determine the crop loss status for the region of interest based on received data such as crop data, field data, production data, weather data, manually ascertained data, geographic data, meteorological data, equipment data, configuration data, optical signature data, or other types of data, as is further described herein. CLDG 116 can output, in response to determining that the crop loss status reflects nonconformance with the acceptable crop loss criteria, at least one alert. For instance, CLDG 116 can output at least one alert via one or more of output devices 126 (e.g., a displayed alert, an audible alert, or other types of alert) and communication devices 124 (e.g., via communication network 112 to computing devices 102).

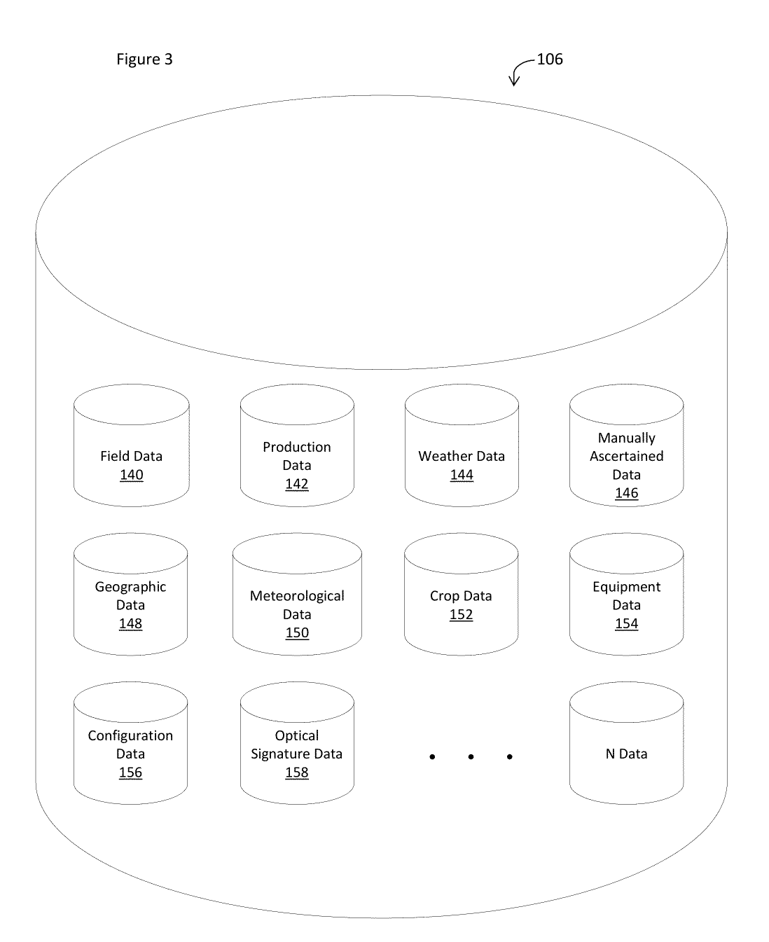

FIG. 3 is a block diagram illustrating further examples of database 106 illustrated in FIG. 1, in accordance with one or more aspects of this disclosure. As illustrated, database 106 can include field data 140, production data 142, weather data 144, manually ascertained data 146, geographic data 148, meteorological data 150, crop data 152, equipment data 154, configuration data 156, and optical signature data 158. In some examples, as is illustrated in FIG. 3 by including “N Data”, database 106 can include one or more types of data that are not illustrated in FIG. 3. That is, the illustration of element “N Data” indicates that data included within database 106 is not limited to the illustrated categories, but may include one or more categories not illustrated in FIG. 3. Similarly, in certain examples, database 106 can include fewer data and/or data categories than are illustrated in FIG. 3. For instance, in some examples, database 106 can include one, two, three, five, or other numbers of data categories, and may not include each of the categories illustrated in FIG. 3. In certain examples, data can be present within database 106 in multiple forms and/or combinations. For instance, in some examples, data can be included in multiple categories of data. In some examples, data can be present within one or more of the categories and represented by multiple forms within the one or more categories.

Field data 140 can include data regarding for example, field locations, the shape of the field, the proximity of the field to other relevant locations such as other fields managed and operated by the user. Field data 140 can, in certain examples, also include field data for the fields of other farmers (e.g., received via a social network or other such method), such as crop quality problems on a nearby field operated by another farmer. For instance, hail damage on another nearby field can indicate a crop quality problem on a neighboring field. In some examples, field data 140 can include data associated with characteristics of the field, such as topographical information, soil types, organic matter, moisture condition and water-carrying capacity, fertility, and other non-crop vegetation on the field. Field data 140 can include data associated with crop conditions over a growing season, such as determined through various sensing methods (e.g., RPVs, in-field sensors, and the like). In certain examples, field data 140 can include data associated with previously performed analyses and determination of crop loss over time.

Production data 142 can include data regarding, for example, crop production practices and/or events. For instance, production data 142 can include historical crop production data associated with a field (APH), including data corresponding to crops planted in prior years and historical yields, including yield maps illustrating yield variability across the field, as-planted maps, and tile maps (e.g., maps indicating locations of drainage tiles installed in the field). As another example, production data 142 can include data associated with historical practices corresponding to a field, such as tillage and irrigation information. Similarly, production data 142 can include data regarding neighboring fields, such as production and/or historical information corresponding to regions physically proximate a region of interest (e.g., a field).

Weather data 144 can include data associated with weather and/or climate data for a region, area, or field. Examples of such information can include, but is not limited to, rainfall data (e.g., average amounts of rainfall, total rainfall for a given period, deviation of precipitation from an average, and the like), hail data (e.g., information corresponding to a hail event, such as a time and location, a size of hail, etc.), temperature data (e.g., average temperatures, deviation of temperature from an average temperature, high temperature within a period of time, low temperature within a period of time, or other temperature data), wind data (e.g., wind speed data, average wind speed data, wind direction data, etc.), or other types of data.

Manually ascertained data 146 can include data relating to knowledge specific to a user and may include, for example, site-specific knowledge, past experiences, activities, observations, and outcomes. For instance, manually ascertained data 146 can include data that is gathered by a user by walking through the crop or inspecting the perimeter of the crop. On some occasions, manually ascertained data 146 can be used (e.g., by CLDG 116) to override or modify an aspect of a crop loss determination analysis, such as by using manually ascertained data 146 rather than corresponding data collected from another source. In some examples, manually ascertained data can include data corresponding to a manual verification of the crop loss determination analysis, such as a manual verification following the issuance of an alert. Such verifications can be gathered during an audit by, for example, a crop insurance adjuster, the farmer manually checking that the scope and extent of the determined crop loss was accurate, and the like.

Geographic data 148 can include geographic data associated with, for example, the region of interest, such as fields included in the region of interest and included in the crop loss determination, analysis, and alerts. Examples of geographic data can include, but are not limited to, geographic data relating to roadways, surface and/or underground water, and landmark locations. Geographic data 148 can be gathered, such as from satellite images, global positioning information, historical information regarding an area of land, plat book service providers, non-governmental and governmental organizations, public and private organizations and agencies, or other sources.

Meteorological data 150 can include data associated with trends in weather and/or climate data for a region of interest over a period of time, such as over weeks, months, years, or other periods of time. For instance, meteorological data 150 can include precipitation data, temperature data, wind speed data, air density data, or other types of meteorological data. In certain examples, meteorological data 150 can include comparisons of such data over a period of time, such as a year-over-year comparison of precipitation data for a region of interest.

Crop data 152 can include information associated with growing crops within a region of interest. For instance, crop data 152 can include data such as a type of seed planted, an average depth at which seeds are planted, a population of seeds planted (e.g., a population density), a time (e.g., a date) when seeds are planted, crop condition data, crop height data, crop color data, crop input data (e.g., types of and/or amounts of fertilizers and/or chemicals applied to the crops), yield estimation data, or other types of data associated with the growing crops within the region of interest.

Equipment data 154 can include information associated with and gathered through the planting, tending, harvesting, crop handling, and storage of crops using equipment during and/or following the growing season. Examples of equipment data may include, but are not limited to, seed location data, seed population data, crop harvesting data (e.g., from yield-monitors included in a combine machine), and data corresponding to weigh scale tickets (i.e., tickets that measure the total amount of crop in a truck, grain tender, or other grain handling equipment).

Configuration data 156 can include configuration data associated with the crop loss analysis. For instance, configuration data 156 can include one or more parameters which, if not satisfied, can trigger CLDG 116 to output at least one alert. Example parameters can include a threshold value, a range of values, or other parameters that CLDG 116 can use to determine whether a crop loss status of growing crops within a region of interest reflects nonconformance with acceptable crop loss criteria. For instance, in examples where one or more of the parameters includes a threshold value, CLDG 116 can compare one or more of the received data for the region of interest with the threshold value, and can determine that the data satisfies the one or more parameters in response to determining that the data is less than the threshold value, less than or equal to the threshold value, greater than the threshold value, greater than or equal to the threshold value, or by other such comparisons. In examples where one or more of the parameters includes a range of values, CLDG 116 can compare one or more of the received data for the region of interest with the range of values, and can determine that the data satisfies the one or more parameters in response to determining that the data is within the range of values. Similarly, CLDG 116 can determine that the data does not satisfy the one or more parameters in response to determining that the data falls outside the range of values.

Optical signature Data 158 can include hyperspectral wavelengths that are determined to correspond to a particular plant under particular conditions. Such conditions can vary from “perfect” plant health to diseased, infested, and/or malnourished plant health. Optical signature data 158 can include the multitude and various hyperspectral wavelengths of a plant, and the corresponding plant and plant environment conditions that result in that hyperspectral wavelength.

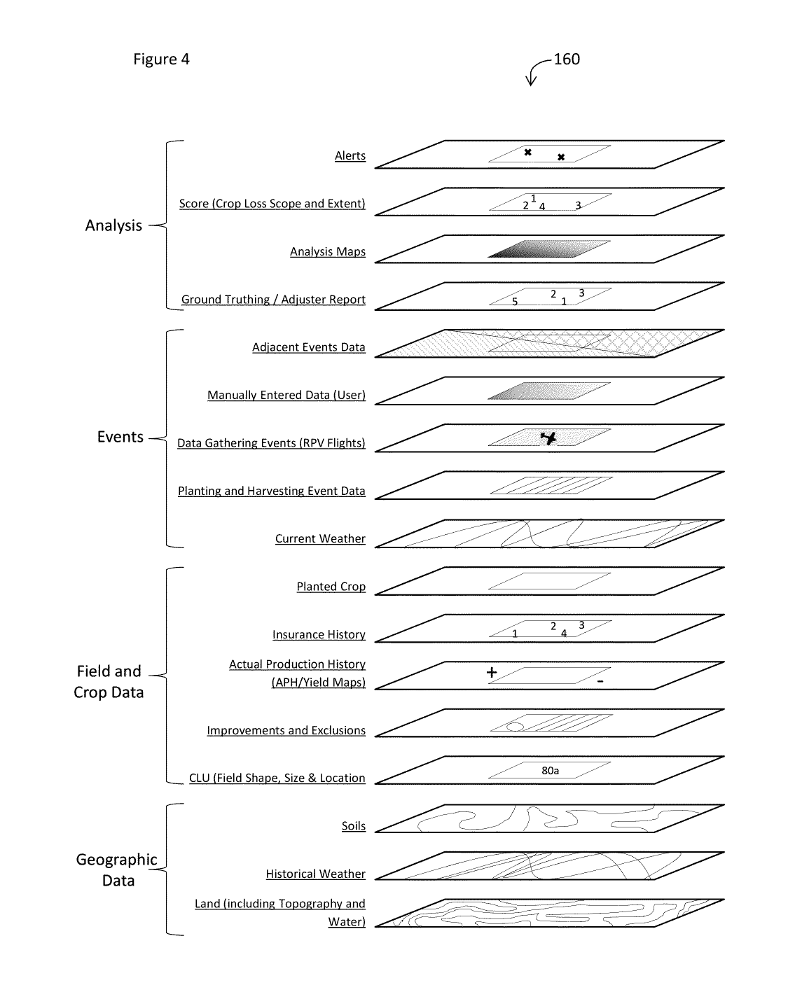

FIG. 4 illustrates an example geographic information system (GIS), in accordance with one or more aspects of this disclosure. As illustrated in FIG. 4, GIS layers image 160 includes multiple data structures, each of which can be regarded as a layer. Such layers can provide information regarding various data elements of a crop loss analysis and alert for a field, including, for example, geographic data, field data, crop data, event data, and analysis data.

Examples of geographic data can include data associated with an area of land (e.g., a field, a field and adjacent areas, and the like). Such data can include topography data, slope data, an indication of the presence of ground water, historical weather and climate data, soil attributes (e.g., soil types, texture, organic matter, fertility test results, etc.), or other types of data. Examples of field and crop data can include the location, size, and shape of the field, data associated with tiling and other improvements made upon the field (e.g., location of tiling and other such improvements), areas of the field to be excluded from crop loss analysis, as well as information related to the field and/or planted crop. In some examples, the field and crop data can include the Actual Production History (APH) and maps for prior years, as well as the insurance history for that field. Examples of event data can include an indication of weather events, such as the occurrence of storms, rain, hail, or excessive wind (e.g., the time, location, and information about such events). Event data can also include human events, such as planting and harvesting event data, and data gathering events, such as RPV flights, the farmer manually entering data of his or her observations, and/or neighboring farmers entering their observations about adjacent fields/areas. Examples of analysis data can include information related to ground truthing or a report and/or audit by the crop insurance adjuster, analysis maps of the condition of the crop, a score of the condition of the crop (including the scope and the extent), and any alerts that have been issued notifying the designated parties that there is a degree of crop loss that exceeds the pre-established parameters.

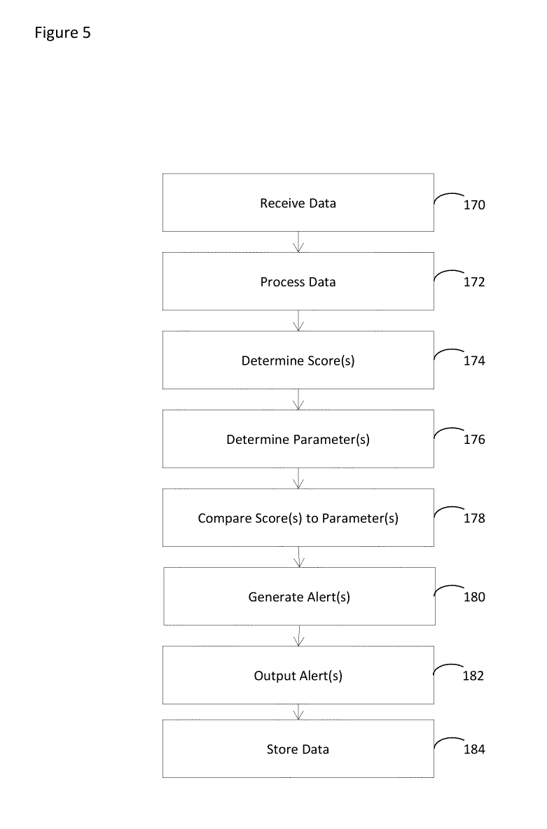

FIG. 5 is a flow diagram illustrating example operations to determine a crop loss status and automatically output at least one alert, in accordance with one or more aspects of this disclosure. For purposes of illustration, the example operations are described below within the context of crop loss determination system 100 and server 104, as shown in FIGS. 1 and 2.

CLDG 116 can receive data for a region of interest that includes growing crops (170). The data for the region of interest can include at least one of field data, crop data, and geographic data. For instance, CLDG 116, executing on one or more processors 120 of server device 104, can receive information from one or more of computing devices 102 (e.g., via user interfaces 114, a social network, etc.), database 106, sensor 108, and data feed 110, such as via communication network 112, point-to-point communications 115, or other such communication methods. Examples of received information can relate to target areas for the crop loss determination system, an RPV data gathering event and the data generated, an in-field sensor, commercial and/or public data, and/or data entered by a user (e.g., via a user interface 114) based on manually ascertained information. Additional examples can include disease and/or pest information that impacts crop quality status from a public or social network or hail, rain, or other weather event that has occurred in the target areas. In some examples, the received data can include one or more previously generated crop loss determination analyses, such as data and/or alerts previously generated by CLDG 116 or another computing system and stored in, for example, database 106. In certain examples, CLDG 116 can receive data for the region of interest from a remote sensor, such as an RPV, as is further described herein.

CLDG 116 can process the received data (172). For example, CLDG 116 can partition the region of interest into a plurality of cells (e.g., a grid). Each cell can represent a portion of the region of interest. The portion (e.g., area) of the region of interest that a cell represents can, in certain examples, be determined based on configuration data (e.g., configuration data 156 illustrated in FIG. 3), such as configuration data received by CLDG 116 from one or more of user interfaces 114. In certain examples, CLDG 116 can partition the region of interest to determine the plurality of cells based on one or more default parameters, such as default parameters stored within configuration data 156. In some examples, CLDG 116 can partition the region of interest to determine the plurality of cells based at least in part on one or more crop loss determination accuracy parameters. For instance, by partitioning the region of interest into smaller cell sizes, CLDG 116 can possibly enable more accurate analyses with respect to each cell, and hence, the entire region of interest.

CLDG 116 can determine one or more scores for the region of interest (174). For example, CLDG 116 can determine one or more scores corresponding to a scope and extent of crop loss within one or more of the plurality of cells and/or corresponding to the entire region of interest. One or more of the scores can, in some examples, be weighted and/or aggregated according to a priority of a category and/or subcategory associated with the received data, as is further described herein.

CLDG 116 can determine one or more parameters corresponding to the received data for the region of interest (176). For instance, the received data can include one or more categories and/or sub-categories. The one or more parameters can, in some examples, represent a value and/or range of values corresponding to acceptable crop loss criteria, such as a range of acceptable precipitation values, temperature values, deviations from averages, and the like. In certain examples, the one or more parameters can represent one or more threshold values, such as maximum and/or minimum values (e.g., minimum precipitation values, maximum wind speed values, or other values).

In some examples, CLDG 116 can change the one or more parameters over the course of, for example, a growing season. For instance, CLDG 116 can automatically adjust one or more of the parameters based on, e.g., an elapsed time of a growing season. In certain examples, CLDG 116 can receive an indication of modified parameters, such as from one or more of user interfaces 114 (e.g., changes that are manually entered by a user, such as a farmer, adjuster, and the like). Accordingly, CLDG 116 can determine the one or more parameters as a function of a sensitivity to generate an alert (e.g., threshold deviation from parameters corresponding to acceptable crop loss criteria), the time of year, the type of crop, the stage of the crop in its growth cycle, and the like. For instance, early in a growing season, CLDG 116 can determine the one or more parameters such that an alert is generated when deviations from parameters associated with acceptable crop loss criteria are smaller in magnitude than later in the growing season. Such changes in the one or more parameters can generate alerts to enable a user (e.g., a farmer) to take reparative actions early in the growing season, while possibly avoiding nuisance alerts later in the growing season.

CLDG 116 can compare the one or more scores to the one or more parameters (178). As an example, CLDG 116 can compare a determined score for a data element of the received data with one or more parameters. In some examples, CLDG 116 can weight and/or aggregate one or more scores to determine a weighted and/or aggregated score for a category and/or sub-category of the received data, as is further described herein.

CLDG 116 can generate, responsive to determining that one or more of the scores reflects nonconformance with acceptable crop loss criteria, at least one alert (180). For example, CLDG 116 can determine that the one or more scores reflects nonconformance with acceptable crop loss criteria based on determining that the one or more scores does not satisfy one or more corresponding parameters. The at least one alert can, in some examples, include an identifier of the region of interest and/or a portion of the region of interest (e.g., cell) that reflects nonconformance with the one or more acceptable crop loss criteria. In certain examples, the at least one alert can include one or more of an indication of a degree by which the region of interest and/or portion of the region of interest deviates from the acceptable crop loss criteria, an indication of a reason for the alert (e.g., an indication of the nonconformance with the acceptable crop loss criteria), a date and/or time of a last data sample, locations of determined change in crop quality, a number of cells excluded from the analysis, a number of cells and/or acres determined to have triggered the alert, a scope of the crop loss, a severity of the crop loss, or other information. In some examples, the at least one alert can include a recommendation for future action for the region of interest, such as a recommendation to “check a field,” a recommendation to maintain surveillance of a field on a “watch list,” a recommendation of a reparative action associated with one or more categories and/or sub-categories of data that reflects nonconformance with the acceptable crop loss criteria, or other recommendations. In some examples, content of the at least one alert can differ based on an identifier of a role of the recipient. For instance, CLDG 116 can output an alert to an insurance agent including information that differs from an alert that is output to a farmer.

CLDG 116 can output the at least one alert (182). For example, CLDG 116 can output the at least one alert, via communication network 112, to one or more of computing devices 102 (e.g., via user interfaces 114). In certain examples, CLDG 116 can output the at least one alert as one or more of a text message, multi-media service (MMS) message, SMS message, voice message, voicemail message, data file, or other types of messages. In certain examples, CLDG 116 can determine a distribution list that includes one or more accounts associated with the region of interest, and can output the at least one alert to each of the accounts included in the list. For instance, the list can include one or more email accounts, telephone numbers, computing device identifiers, and the like, that can, in certain examples, be associated with one or more users. Examples of such users can include, but are not limited to, farmers, crop insurance agents, crop insurance adjusters, agricultural product buyers, agricultural landlords, agricultural bankers, or other such users. In this way, CLDG 116 can output at least one alert that can notify one or more users that the determined crop loss status reflects nonconformance with the acceptable crop loss criteria.

CLDG 116 can store data associated with the crop loss status analysis (184). For instance, CLDG 116 can store data (e.g., within database 106) associated with the one or more parameters, received data that reflects nonconformance with the acceptable crop loss criteria, the extent by which the received data reflects the nonconformance, or other data. Accordingly, CLDG 116 can use such data during subsequent analyses. That is, the described operations of FIG. 5 can be iterative in nature, such that CLDG 116 receives data, performs operations described with respect to FIG. 5, generates one or more alerts and stores data, and uses such stored data in future iterations of the operations. In this way, CLDG 116 can possibly improve the accuracy of subsequent analyses based on prior determinations and iterations of the operations.

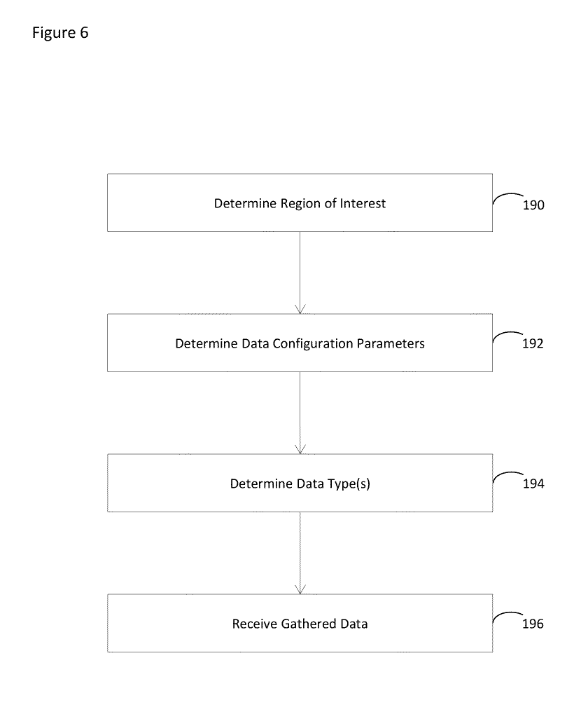

FIG. 6 is a flow diagram illustrating further details of operation 170 as shown in FIG. 5, in accordance with one or more aspects of this disclosure. CLDG 116 can determine a region of interest (190). For instance, CLDG 116 can receive configuration parameters (e.g., via one or more of user interfaces 114) that define the boundaries (e.g., physical boundaries, such as latitude and longitude data) of the region of interest. In some examples, the region of interest can include a field (e.g., a field of growing crops). In other examples, the region of interest can include one or more portions of a field of growing crops. For instance, a user can define a portion of the field to be analyzed and/or portions of the field that are not to be analyzed. Such portions of a field that are not to be analyzed can be referred to as exclusion zones, and can correspond to regions associated with physical features such as build sites, prior build sites, areas of prior manure spills, or other regions that are not to be included in the crop loss determination analysis.

CLDG 116 can determine data configuration parameters corresponding to the region of interest (192). For instance, CLDG 116 can determine the number, size, and/or location of boundaries by which to partition the region of interest to determine a plurality of cells, each of the cells representing a portion of the region of interest. Such cell boundary information can be determined by CLDG 116 (e.g., based on default parameters) and/or received by CLDG 116, such as from one or more of user interfaces 114.

CLDG 116 can determine one or more data types included in the received data for the region of interest (194). As an example, CLDG 116 can receive an indication of the one or more data types from one or more of user interfaces 114. CLDG 116 can receive gathered data for the region of interest (196). For instance, CLDG 116 can receive data for the region of interest from one or more of sensor 108 (e.g., one or more remote sensors, such as an RPV, a satellite, an aircraft, and the like), data feed 110, database 106, and computing devices 102.

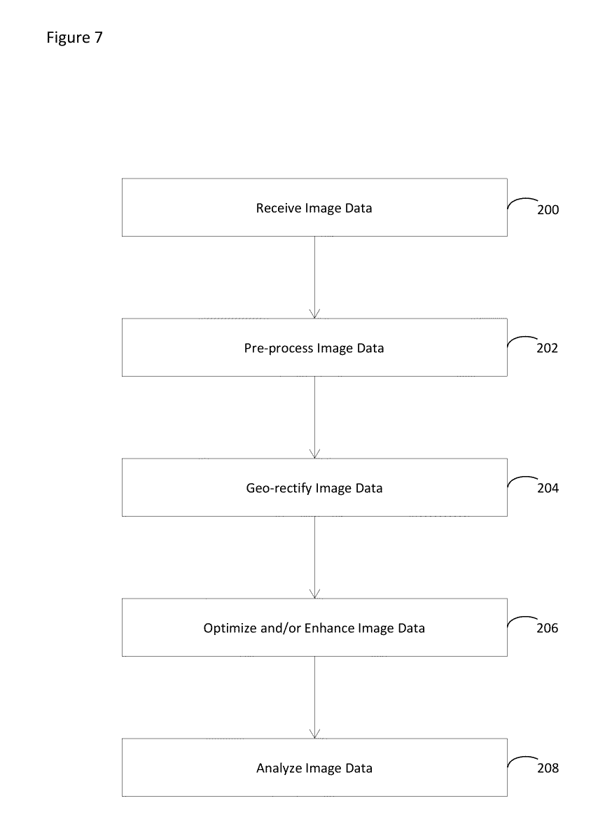

FIG. 7 is a flow diagram illustrating further details of operation 170 as shown in FIG. 5, in accordance with one or more aspects of this disclosure. FIG. 7 illustrates example operations of CLDG 116 to receive and analyze image data according to techniques of this disclosure. CLDG 116 can receive image data for the region of interest (200). For instance, CLDG 116 can receive image data, such as visible-spectrum image data, ultra-violet image data, infrared image data, hyperspectral wavelength image data, or other types of image data. In certain examples, CLDG 116 can receive the image data in the form of multiple image files, each of the image files corresponding to a different sub-region of the region of interest.

CLDG 116 can pre-process the received image data (202). For example, CLDG 116 can assemble (e.g., “stitch”) the multiple image files together to generate an image file corresponding to the entire region of interest. CLDG 116 can, in some examples, pre-process the image data to discard image data that is not associated with the region of interest or is below a threshold quality (e.g., a threshold clarity, brightness, contrast, and the like).

CLDG 116 can geo-rectify the image data (204). For example, CLDG 116 can associate portions of the pre-processed image data with latitude and longitude values corresponding to known latitude and longitude values that the portion of the image represents. CLDG 116 can optimize and/or enhance the geo-rectified image data (206). For instance, CLDG 116 can adjust a brightness, contrast, or other image parameters to enhance one or more of the image parameters (e.g., to make a boundary and/or image of crop loss more visually apparent). CLDG 116 can analyze the image data (208). As an example, CLDG 116 can juxtapose the geo-rectified image against previous images of the same crop to determine a change in the crop loss status and/or growth status over time.

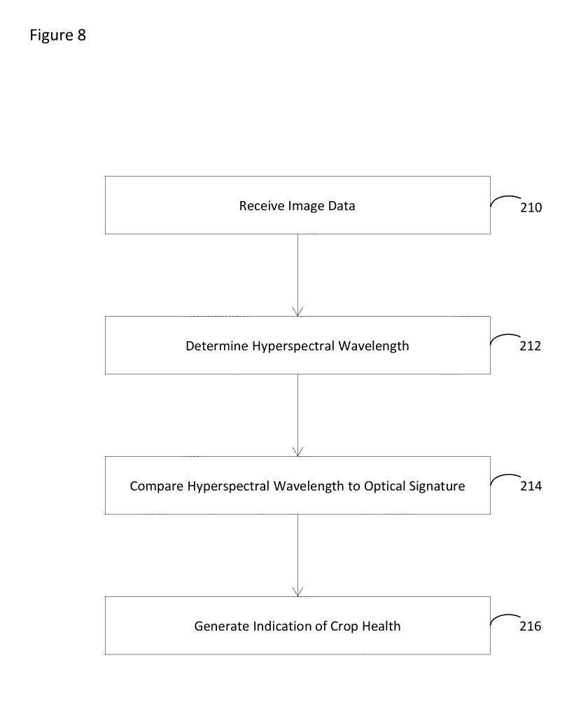

FIG. 8 is a flow diagram illustrating further details of operation 172 as shown in FIG. 5, in accordance with one or more aspects of this disclosure. In particular, FIG. 8 illustrates example operations of CLDG 116 to generate an indication of crop health using hyperspectral wavelength data. CLDG 116 can receive image data for the region of interest (210). For example, CLDG 116 can receive image data from an in-field image sensor (e.g., included in a camera device) and/or remote image sensor, such as from a camera device carried by one or more of an RPV, an aircraft, and a satellite. Crop loss determination generator 116 can determine hyperspectral wavelength data from the received image data (212). CLDG 116 can compare the hyperspectral wavelength data to one or more optical signatures (214). Using the optical signatures, CLDG 116 can determine an indication of crop health based on the comparison (216).

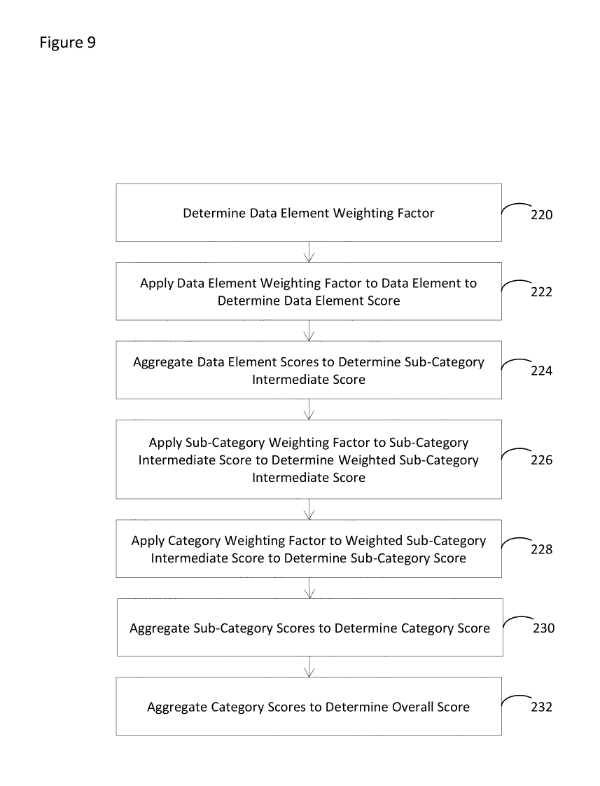

FIG. 9 is a flow diagram illustrating further details of operation 174 as shown in FIG. 5, in accordance with one or more aspects of this disclosure. CLDG 116 can determine a data element weighting factor corresponding to a data element of received data for the region of interest (220). For instance, CLDG 116 can access configuration data (e.g., stored in database 106) to determine a weighting factor associated with the data element, as is further described herein. CLDG 116 can apply the data element weighting factor to the data element to determine a data element score (222). For example, CLDG 116 can multiply a value of the data element by a value of the weighting factor to determine the data element score.

CLDG 116 can aggregate data element scores to determine a sub-category intermediate score (224). For instance, the received data for the region of interest can include one or more categories. Examples of categories can include, but are not limited to, drought data, sensor data, land data (including topography and water data), historical weather data, soil data, field data (e.g., field shape, size, and location), improvements data (e.g., improvements to the region of interest, such as addition of drain tile or other improvements), production history data, insurance claim history data, planted crop data, planting and harvesting event data, manually entered data, adjacent event data (e.g., weather events such as hail, disease, infestation, or other events associated with a location proximate a region of interest), adjuster report data, or other categories of data. At least one of the categories can include one or more sub-categories. For instance, a drought data category can include sub-categories such as night time high temperatures, day time high temperatures, relative humidity, season precipitation deviation from average, two week precipitation deviation from average, average interval of days between rainfall of three-tenths of one inch, or other sub-categories. CLDG 116 can aggregate the data element scores within sub-categories to determine sub-category intermediate scores for the sub-categories. As one example, CLDG 116 can aggregate the data element scores by summing the data element scores. In other examples, CLDG 116 can aggregate the data element scores by multiplying, averaging, or by using other aggregation techniques.

CLDG 116 can apply a sub-category weighting factor to the sub-category intermediate score to determine a weighted sub-category intermediate score (226). CLDG 116 can apply a category weighting factor to the weighted sub-category intermediate score to determine a sub-category score (228). CLDG 116 can aggregate sub-category scores to determine a category score (230). CLDG 116 can aggregate category scores to determine an overall score (232). CLDG 116 can determine the overall score with respect to an entire region of interest, a portion of the region of interest (e.g., a cell), or both.

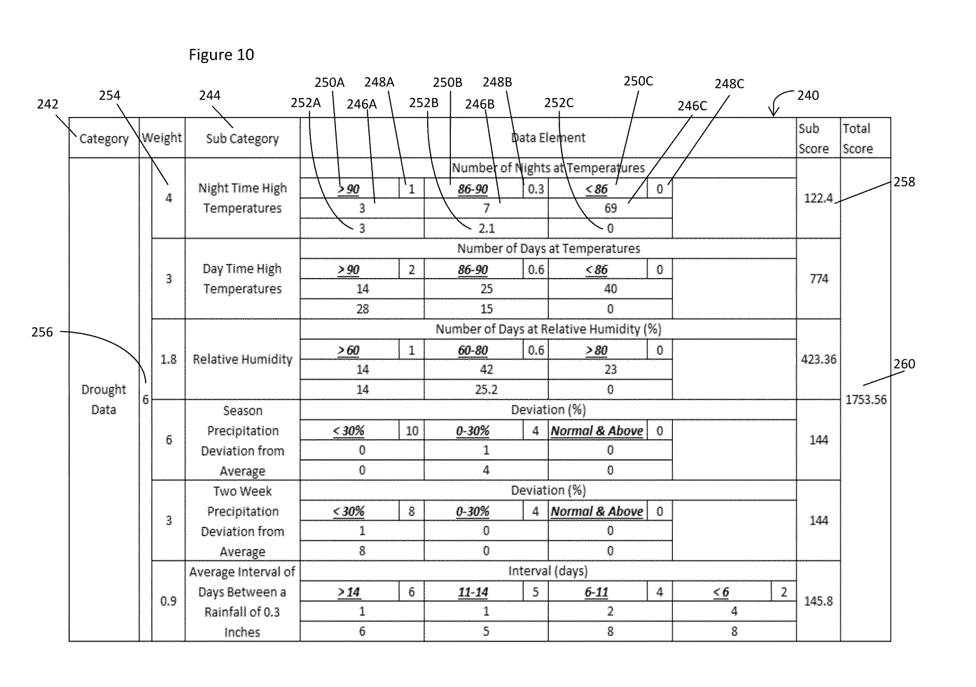

FIG. 10 illustrates a table 240 that represents an example scoring matrix for use in a method of determining a crop loss status of growing crops within a region of interest, in accordance with one or more aspects of this disclosure. As illustrated in FIG. 10, table 240 can include category 242 of received data for a region of interest. However, while illustrated with respect to one category, in certain examples, table 240 can include a plurality of categories, such as two categories, three categories, ten categories, or other numbers of categories. In the illustrated example, category 242 corresponds to drought data. Other example categories can include, but are not limited to, sensor data, event data, land data, historical weather data, soils data, field data, improvements data, production history data, insurance claim history data, planted crop data, planting and harvesting event data, manually entered data, adjacent event data, adjuster report data, or other categories of data.

As further illustrated in FIG. 10, category 242 can include sub-categories 244, including night time high temperatures, day time high temperatures, relative humidity, season precipitation deviation from average, two week precipitation deviation from average, and average interval of days between rainfalls of three-tenths of one inch. In certain examples, sub-categories 244 can include more or fewer sub-categories. In general, sub-categories 244 can include any number of sub-categories (e.g., zero, one, two, five, fifty, or other numbers of sub-categories) that are deemed relevant to a category of data.

CLDG 116 can classify received data for the region of interest according to a sub-category and/or category. Received data can take the form of a data element, such as data elements 246A-246C. CLDG 116 can determine a data element weighting factor for each of the one or more data elements, such as data element weighting factors 248A-248C. In some examples, CLDG 116 can determine the data element weighting factors for each of the one or more data elements based on a comparison of the data element to one or more threshold values. For instance, as illustrated in FIG. 10, CLDG 116 can determine that data element weighting factor 248A is to be applied to data element 246A based on a comparison of data element 246A with threshold value 250A. Similarly, CLDG 116 can determine that data element weighting factor 248B is to be applied to data element 246B based on a comparison of data element 246B with threshold values 250B (i.e., a range of threshold values). CLDG 116 can determine that data element weighting factor 248C is to be applied to data element 246C based on a comparison of data element 246C with threshold value 250C. In this way, as illustrated in FIG. 10, CLDG 116 can determine a plurality of data element weighting factors to be applied to a plurality of data elements corresponding to a plurality of sub-categories within the category. Similarly, CLDG 116 can determine such data element weighting factors for a plurality of sub-categories within a plurality of categories.

CLDG 116 can apply the determined data element weighting factors (e.g., data element weighting factors 248A-248C) to the data elements (e.g., data elements 246A-246C) to determine a plurality of data element scores, such as data element scores 252A-252C. For example, CLDG 116 can multiply data element 246A by weighting factor 248A to determine data element score 252A. Similarly, CLDG 116 can multiply data element 246B by weighting factor 248B to determine data element score 252B, and can multiply data element 246C by weighting factor 248C to determine data element score 252C.

CLDG 116 can aggregate (e.g., sum, multiply, average, and the like) the data element scores (e.g., data element scores 252A-252C) to determine a sub-category sub-score. For instance, CLDG 116 can sum data element scores 252A-252C to determine the sub-category sub-score (e.g., summing by the equation “3+2.1+0” to determine a sub-score of “5.1”). CLDG 116 can apply a sub-category weighting factor, such as sub-category weighting factor 254 to determine a sub-category intermediate score. For instance CLDG 116 can multiply sub-category weighting factor 254 by the determined sub-category sub-score (e.g., “5.1” in this example) to determine a sub-category intermediate score (e.g., “20.4” in this example). CLDG 116 can apply (e.g., multiply) a category weighting factor, such as category weighting factor 256, to the determined sub-category intermediate score to determine a sub-category score for the sub-category. For instance, CLDG 116 can multiply category weighting factor 256 (e.g., “6” in this example) by the determined sub-category intermediate score (e.g., “20.4” in this example) to determine subcategory score 258 (e.g., “122.4” in this example). As illustrated, CLDG 116 can determine a plurality of sub-category scores for a plurality of sub-categories. CLDG 116 can aggregate the sub-category scores to determine a category score, such as category score 260. In some examples, CLDG 116 can aggregate a plurality of determined category scores to determine an overall score. For instance, CLDG 116 can determine an overall score (e.g., for a portion of a region of interest such as a cell, for the entire region of interest, or for other areas) as the sum of a plurality of determined category scores.

Each of the above-described weighting factors (i.e., data element weighting factors, sub-category weighting factors, and category weighting factors) can be different or the same. In addition, each of the weighting factors can be modified, such as automatically by CLDG 116 and/or in response to input received from one or more of user interfaces 114. For instance, a user can modify one or more of the weighting factors, such as by providing user input via one or more of user interfaces 114 to adjust a weighting factor and/or provide a new value for the weighting factor.

The scoring matrix represented by table 240 can be associated with a portion of a region of interest (e.g., a cell), an entire region of interest (e.g., a field), or both. CLDG 116 can compare one or more of the determined scores and/or values within table 240 with one or more parameters corresponding to acceptable crop loss criteria to determine whether the crop loss status reflects nonconformance with the acceptable crop loss criteria. As one example, CLDG 116 can compare one or more of the data element scores with one or more parameters, and can output at least one alert in response to determining that one or more of the data element scores does not satisfy the one or more parameters (and therefore reflects nonconformance with the acceptable crop loss criteria). As another example, CLDG 116 can compare one or more of the sub-category scores with the one or more parameters, and can output at least one alert in response to determining that one or more of the sub-category scores does not satisfy the one or more parameters. As yet another example, CLDG 116 can compare one or more of the category scores and/or total score with the one or more parameters, and can output at least one alert in response to determining that one or more of the category scores and/or total score does not satisfy the one or more parameters.

Accordingly, CLDG 116 can determine a crop loss status for a region of interest at a level of granularity based on a size of a cell of the region of interest, or for the region of interest as a whole. CLDG 116 and/or a user (e.g., via user interfaces 114) can modify one or more of the parameters and/or the weighting factors, thereby modifying a level of sensitivity of the generation of alerts and/or a contribution of one or more forms of data to the generation of alerts.

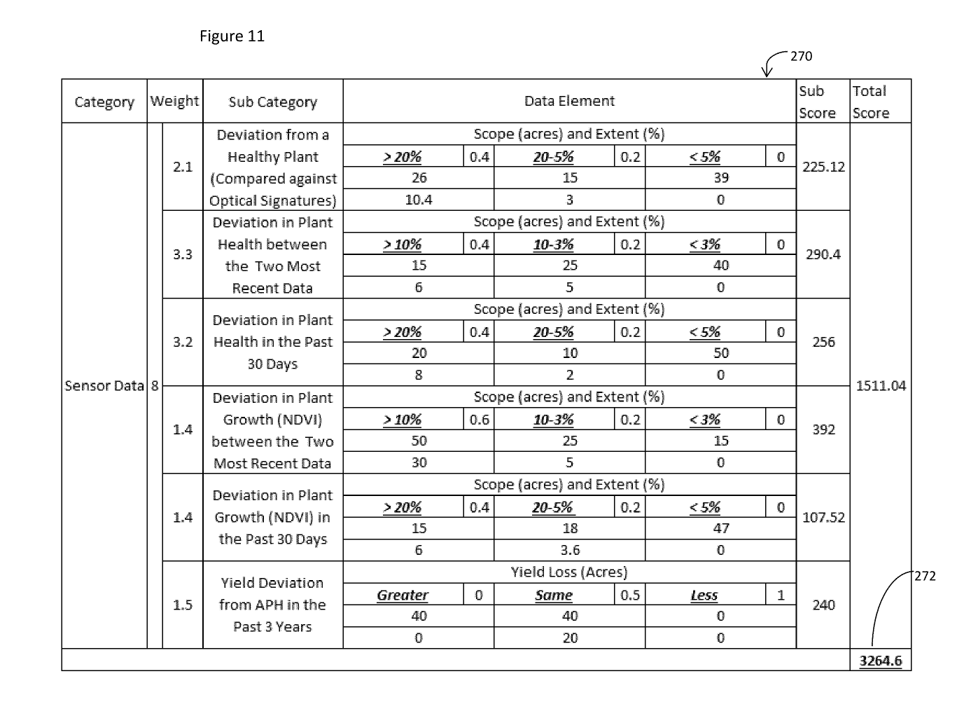

FIG. 11 illustrates table 270 that represents an example scoring matrix for use in a method of determining a crop loss status of growing crops within a region of interest, in accordance with one or more aspects of this disclosure. Specifically, FIG. 11 illustrates table 270 that represents an example scoring matrix with respect to different (i.e., as compared to table 240 of FIG. 10) categories and sub-categories of data. As illustrated in FIG. 11, CLDG 116 can receive data for a category of sensor data. The sensor data category can include a plurality of sub-categories, such as sub-categories corresponding to deviation from a healthy plant (compared against optical signatures), deviation in plant health between the two most recent data capture events, deviation in plant health in the past thirty days, deviation in plant growth (NDVI) between the two most recent data capture events, deviation in plant growth (NDVI) in the past thirty days, and yield deviation from APH in the past three years. As illustrated, CLDG 116 can determine one or more data element weighting factors and apply the determined data element weighting factors to the data elements to determine one or more data element scores. CLDG 116 can aggregate the one or more data element scores within a sub-category to determine a sub-category sub-score. CLDG 116 can apply a sub-category weighting factor to the sub-category sub-score to determine a sub-category intermediate score, and can apply a category weighting factor to the sub-category intermediate score to determine a sub-category score. CLDG 116 can aggregate the sub-category scores to determine one or more category scores. In some examples, CLDG 116 can aggregate the category scores to determine overall score 272, such as an overall score for a portion of a region of interest (e.g., a cell) and/or the entire region of interest.

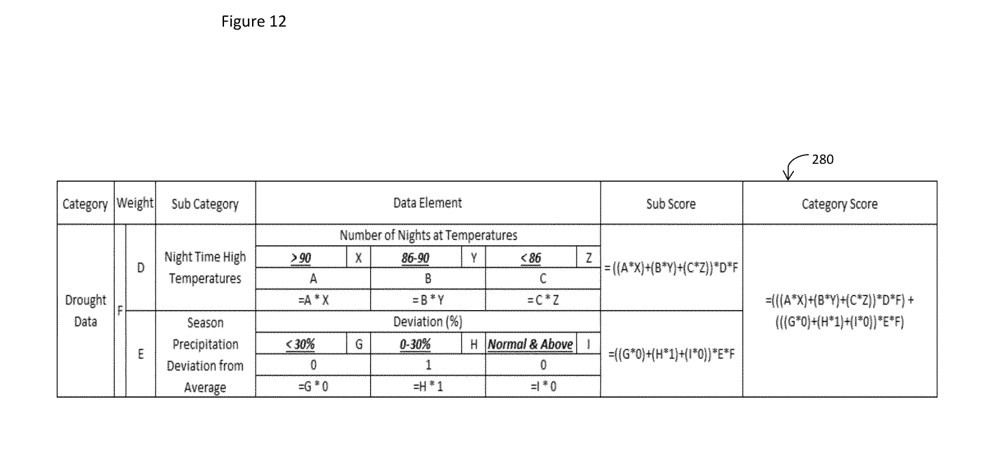

FIG. 12 illustrates table 280 that represents example calculations that can be used to determine a crop loss status of growing crops within a region of interest, in accordance with one or more aspects of this disclosure. Specifically, table 280 further illustrates example calculations as described above with respect to FIG. 10 that can be used to determine data element scores, sub-category scores, and a category score.

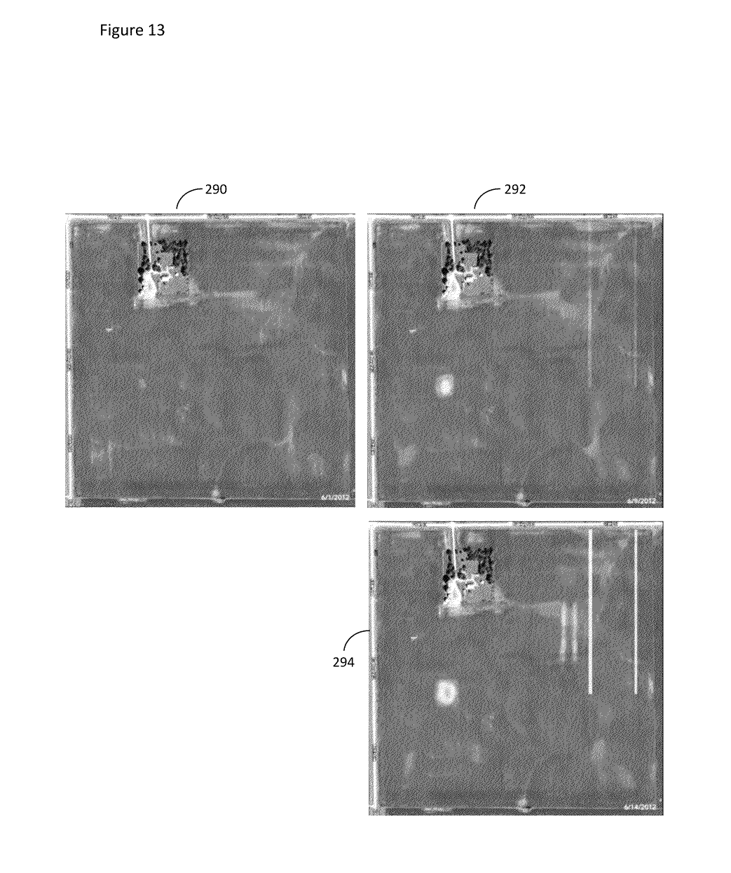

FIG. 13 illustrates example images 290, 292, and 294 that can be used to determine a crop loss status for a region of interest, in accordance with one or more aspects of this disclosure. Images 290, 292, and 294 represent example image data that can be received by CLDG 116 (e.g., via an RPV). Images 290, 292, and 294 represent images of a field captured over a period of days (e.g., image 290 captured at a first time, image 292 captured at a second, later time, and image 294 captured at a third time, later than the second time). Images 290, 292, and 294 illustrates changes in the condition of the crop over time. CLDG 116 can analyze images 290, 292, and 294, and can determine the crop loss status based at least in part on the analysis. As described above, the crop loss status can be determined, for example, based at least in part on texture, color (traditional, infrared, etc.), patterns, tone, shadows, and temperature combined with other available data. Images 290, 292, and 294 are examples of image data that the CLDG 116 can receive. In some examples, certain visual and other display techniques can be used to make the crop quality deficiencies more visually apparent from the images. For instance, CLDG 116 can amplify visual indicators of the growth of the crop by electronic means to enhance the image and illustrate any deficiencies in a more visually apparent manner. As another example, CLDG 116 can use time-lapse techniques, such that changing crop conditions can be visually observed over time through the use of multiple images juxtaposed together.

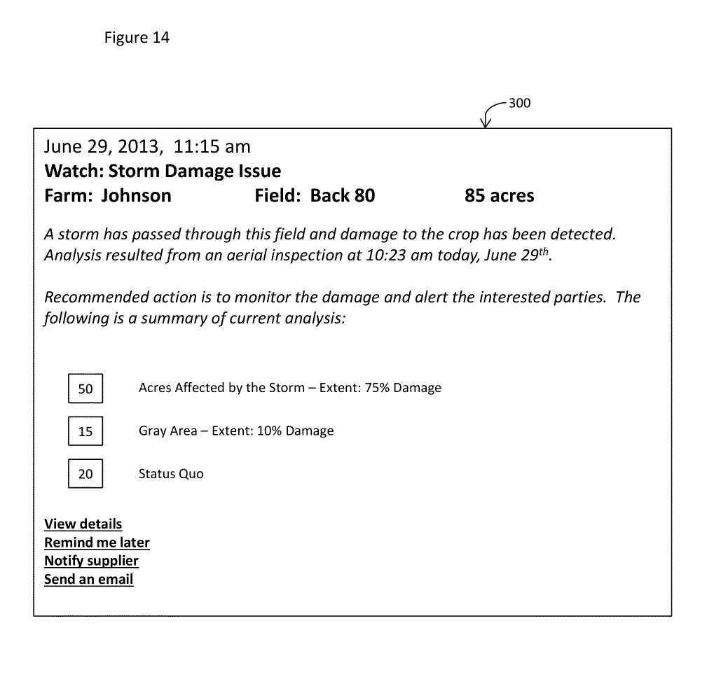

FIG. 14 illustrates an example user interface 300 including an alert, in accordance with one or more aspects of this disclosure. User interface 300 is an example user interface that can be output, for display (e.g., at one or more of user interfaces 114), by CLDG 116. FIG. 14 illustrates an example alert output by CLDG 116 after determination that a storm has passed through a field, and after an aerial inspection with the use of an RPV. In this example, CLDG 116 determines that seventy-five percent damage of the region of interest has been confirmed on fifty acres of the region of interest, with fifteen acres determined to be in the “gray area” and having ten percent damage, and twenty acres confirmed to not have been damaged.

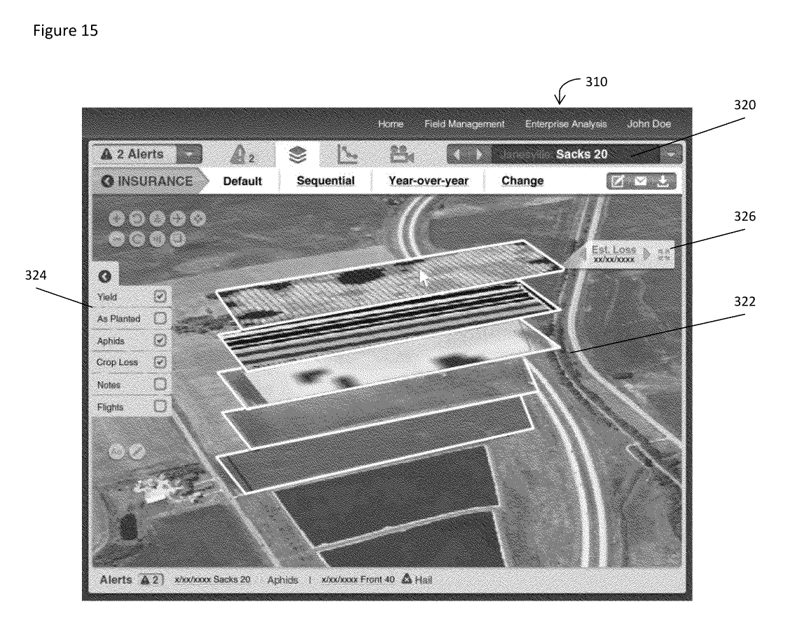

FIG. 15 illustrates an example user interface 310 that can be used to review information related to crop loss, in accordance with one or more aspects of this disclosure. In the example of FIG. 15, a user has flown an RPV and uploaded the gathered data into CLDG 116 in order to view a crop insurance analysis. As illustrated, user interface 310 can enable the user can access such information regardless of whether or not an alert has been triggered. It should also be noted that the content of user interface 310 can vary depending on the role of the user (e.g., farmer, insurance agent, insurance adjuster, landlord, banker, or other roles) and the presentation of the content can vary depending on the manner in which it is viewed.

User interface 310 outputs (e.g., displays) information regarding crop loss determination for a specific field along with additional information that may be helpful to a user. In this example, field identifiers 320 as well as multiple “stacked” images of the field 322 can be output. Along with the field images 320, there can be a modifiable field view area 324 that includes graphical controls that can allow the user to alter the views of the field 322, in addition to the ability to exclude areas of the field that are not to be included in the analysis. An analysis area 326 can be output including identifiers of the acres included in the region of interest, the current estimated loss for particular areas (e.g., percentages) of the region of interest, the actual production history (APH) of the region of interest, the current estimated yield for the region of interest, or other such information.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method comprising:
receiving, by a crop loss determination generator (CLDG) executing on a computing device, data for a region of interest that includes growing crops, wherein the data for the region of interest comprises at least one of field data, crop data, and geographic data;
determining, by the CLDG and based on the received data for the region of interest, that a crop loss status of the growing crops within the region of interest reflects nonconformance with acceptable crop loss criteria, wherein the crop loss status comprises an indication of at least one of an extent and a scope of crop loss of the growing crops within the region of interest; and
outputting, by the CLDG and in response to determining that the crop loss status reflects nonconformance with the acceptable crop loss criteria, at least one alert.

2. The method of claim 1, wherein the region of interest comprises a plurality of cells, and wherein determining that the crop loss status of the growing crops within the region of interest reflects nonconformance with the acceptable crop loss criteria further comprises:
determining, by the CLDG, a crop loss score for one or more of the plurality of cells of the region of interest to determine one or more crop loss cell scores;
aggregating, by the CLDG, the one or more crop loss cell scores to determine a crop loss score for the region of interest; and
determining, by the CLDG, that the crop loss score for the region of interest does not satisfy one or more parameters corresponding to the acceptable crop loss criteria.

3. The method of claim 1, wherein the region of interest comprises a plurality of cells, and wherein determining that the crop loss status of the growing crops within the region of interest reflects nonconformance with the acceptable crop loss criteria further comprises:
determining, by the CLDG, a crop loss score for at least one cell from the plurality of cells; and
determining, by the CLDG, that the crop loss score for the at least one cell does not satisfy one or more parameters corresponding to the acceptable crop loss criteria.

4. The method of claim 3, wherein the data for the region of interest comprises one or more categories, and wherein determining the crop loss score for the at least one cell from the plurality of cells comprises:
determining, by the CLDG, a category score for each of the one or more categories to determine one or more category scores for the at least one cell; and
aggregating, by the CLDG, the one or more category scores to determine the crop loss score for the at least one cell.

5. The method of claim 4, wherein determining the one or more category scores for the at least one cell further comprises:
applying, by the CLDG, a category weighting factor to each of the one or more category scores to determine the one or more category scores for the at least one cell.

6. The method of claim 5, wherein applying the category weighting factor to each of the one or more category scores comprises:
applying, by the CLDG, a first category weighting factor to a first one of the one or more category scores to determine a first category score for the at least one cell; and
applying, by the CLDG, a second category weighting factor to a second one of the one or more category scores to determine a second category score for the at least one cell,
wherein the second category weighting factor is different than the first category weighting factor.

7. The method of claim 4, wherein at least one of the one or more categories for the at least one cell comprises one or more sub-categories, and wherein determining the one or more category scores for the at least one cell comprises:
determining, by the CLDG, a sub-category score for each of the one or more sub-categories to determine one or more sub-category scores for the one or more categories; and
aggregating, by the CLDG, the one or more sub-category scores to determine the one or more category scores for the at least one cell.

8. The method of claim 7, wherein determining the one or more sub-category scores for the one or more categories further comprises:
applying, by the CLDG, a sub-category weighting factor to each of the one or more sub-category scores to determine one or more sub-category intermediate scores; and
applying, by the CLDG, the category weighting factor to the one or more sub-category intermediate scores to determine the one or more sub-category scores.

9. The method of claim 8, wherein applying the sub-category weighting factor to each of the one or more sub-category scores to determine the one or more sub-category intermediate scores comprises:
applying, by the CLDG, a first sub-category weighting factor to a first one of the one or more sub-category scores to determine a first sub-category intermediate score; and
applying, by the CLDG, a second sub-category weighting factor to a second one of the one or more sub-category scores to determine a second sub-category intermediate score,
wherein the second sub-category weighting factor is different than the first sub-category weighting factor.

10. The method of claim 7, wherein at least one of the one or more sub-categories comprises one or more data elements, and wherein determining the one or more sub-category scores for the one or more categories comprises:
determining, by the CLDG, a data element score for each of the one or more data elements to determine one or more data element scores for the one or more sub-categories; and
aggregating, by the CLDG, the one or more data element scores to determine the one or more sub-category scores for the one or more categories.

11. The method of claim 10, wherein determining the one or more data element scores for the one or more sub-categories further comprises:
determining, by the CLDG, a data element weighting factor for each of the one or more data elements; and
applying, by the CLDG, the data element weighting factor to each of the one or more data elements to determine the one or more data element scores.

12. The method of claim 11, wherein applying the data element weighting factor to each of the one or more data element scores to determine the one or more data element scores for the one or more sub-categories comprises:
applying, by the CLDG, a first data element weighting factor to a first one of the one or more data element scores to determine a first data element score for the one or more sub-categories; and
applying, by the CLDG, a second data element weighting factor to a second one of the one or more data element scores to determine a second data element score for the one or more sub-categories,
wherein the second data element weighting factor is different than the first data element weighting factor.

13. The method of claim 3, further comprising partitioning, by the CLDG, the region of interest to determine the plurality of cells.

14. The method of claim 3, wherein the at least one alert comprises an identifier of the at least one cell that does not satisfy the one or more parameters corresponding to the acceptable crop loss criteria.

15. The method of claim 1, wherein the at least one alert comprises one or more of a short messaging service (SMS) message, a multi-media service (MMS) message, an email message and a telephonic message.

16. The method of claim 1, further comprising determining, by the CLDG, an alert distribution list that comprises a plurality of account identifiers, wherein outputting the at least one alert comprises outputting a first alert to a first account associated with a first one of the plurality of account identifiers and outputting a second alert to a second account associated with a second one of the plurality of account identifiers.

17. The method of claim 1, wherein the at least one alert comprises an indication of a degree by which the crop loss status deviates from the acceptable crop loss criteria.

18. The method of claim 1, wherein receiving the data for the region of interest comprises receiving data for the region of interest collected from one or more sensors.

19. The method of claim 18, wherein the one or more sensors are carried by at least one of an aerial vehicle and a satellite.

20. The method of claim 18, wherein the one or more sensors comprise at least one of a field sensor and a remote sensor.

21. The method of claim 1, wherein receiving the data for the region of interest comprises receiving image data for the region of interest.

22. The method of claim 21, wherein the image data comprises at least one of crop color data, crop texture data, crop pattern data, and hyperspectral wavelength data of the growing crops within the region of interest.

23. The method of claim 22, wherein the image data comprises hyperspectral wavelength data of the growing crops within the region of interest, and wherein determining that the crop loss status of the growing crops within the region of interest reflects nonconformance with the acceptable crop loss criteria further comprises:
comparing, by the CLDG, the hyperspectral wavelength data of the growing crops within the region of interest with one or more optical signatures; and
determining, by the CLDG, that the crop loss status of the growing crops within the region of interest reflects nonconformance with the acceptable crop loss criteria based at least in part on the comparison of the hyperspectral wavelength data with the one or more optical signatures.

24. The method of claim 1, wherein receiving the data for the region of interest comprises receiving the data for the region of interest from a user interface communicatively coupled to the computing device.

25. The method of claim 1, wherein receiving the data for the region of interest comprises receiving the data for the region of interest from a database communicatively coupled to the computing device and configured to store at least one of the field data, the crop data, and the geographic data.

26. A system comprising:
a computing device comprising at least one processor; and
a crop loss determination generator (CLDG) executable by the at least one processor of the computing device and configured to:
receive data for a region of interest that includes growing crops, wherein the data for the region of interest comprises at least one of field data, crop data, and geographic data;
determine, based on the received data for the region of interest, that a crop loss status of the growing crops within the region of interest reflects nonconformance with acceptable crop loss criteria, wherein the crop loss status comprises an indication of at least one of an extent and a scope of crop loss of the growing crops within the region of interest; and
output, in response to determining that the crop loss status reflects nonconformance with the acceptable crop loss criteria, at least one alert.

27. The system of claim 26, wherein the region of interest comprises a plurality of cells, and wherein the CLDG is configured to determine that the crop loss status of the growing crops within the region of interest reflects nonconformance with the acceptable crop loss criteria by at least being configured to:
determine a crop loss score for one or more of the plurality of cells of the region of interest to determine one or more crop loss cell scores;
aggregate the one or more crop loss cell scores to determine a crop loss score for the region of interest; and
determine that the crop loss score for the region of interest does not satisfy one or more parameters corresponding to the acceptable crop loss criteria.

28. The system of claim 26, wherein the region of interest comprises a plurality of cells, and wherein the CLDG is configured to determine that the crop loss status of the growing crops within the region of interest reflects nonconformance with the acceptable crop loss criteria by at least being configured to:
determine a crop loss score for at least one cell from the plurality of cells; and
determine that the crop loss score for the at least one cell does not satisfy one or more parameters corresponding to the acceptable crop loss criteria.

29. The system of claim 26, wherein the CLDG is configured to receive data for the region of interest by at least being configured to receive data for the region of interest collected from one or more sensors.

30. The system of claim 29, wherein the one or more sensors are carried by at least one of an aerial vehicle and a satellite.

31. The system of claim 29, the CLDG is configured to receive data for the region of interest by at least being configured to receive image data for the region of interest that comprises at least one of crop color data, crop texture data, crop pattern data, and hyperspectral wavelength data of the growing crops within the region of interest.

32. A computer-readable storage medium encoded with instructions that, when executed, cause at least one processor of a computing device to:
receive data for a region of interest that includes growing crops, wherein the data for the region of interest comprises at least one of field data, crop data, and geographic data;
determine, based on the received data for the region of interest, that an agronomic status of the growing crops within the region of interest reflects nonconformance with acceptable criteria, wherein the agronomic status comprises an indication of at least one of an extent and a scope of crop loss of the growing crops within the region of interest; and
output, in response to determining that the agronomic status reflects nonconformance with the acceptable criteria, at least one alert.

33. A method comprising:
receiving, by a field growth determination generator (FGDG) executing on a computing device, data for a region of interest that includes growing biological matter, wherein the data for the region of interest comprises at least one of field data, agronomic data, and geographic data;
determining, by the FGDG and based on the received data for the region of interest, that an agronomic status of the biological matter within the region of interest reflects nonconformance with acceptable criteria; and
outputting, by the FDDG and in response to determining that the agronomic status reflects nonconformance with the acceptable criteria, at least one alert.
